(19) United States
(12) Patent Application Publication
(10) Pub. No.: US 2026/0192114 A1
(43) Pub. Date: Jul. 9, 2026

(54) METHOD AND SYSTEM TO STIMULATE PHRENIC NERVE TO TREAT SLEEP APNEA

(71) Applicant: Lunair Medical, Inc., Minneapolis, MN (US)

(72) Inventors: Alan SCHWARTZ, Baltimore, MD (US); Hartmut SCHNEIDER, Eindhoven (NL); Zoar ENGELMAN, New York, NY (US); Edward RUPPEL, New York, NY (US); Mark GELFAND, Porto (PT)

(21) Appl. No.: 19/130,888

(22) PCT Filed: Nov. 17, 2023
(86) PCT No.: PCT/US2023/080199

§ 371 Date: May 16, 2025

Related U.S. Application Data
(60) Provisional application No. 63/426,072, filed on Nov. 17, 2022.
(60) Provisional application No. 63/442,331, filed on Jan. 31, 2023.

(51) Int. Cl. A61N 1/36; A61B 5/00; A61B 5/08

(52) U.S. Cl. CPC A61N 1/36139; A61B 5/08; A61B 5/4809; A61B 5/4818; A61B 5/4836; A61N 1/3601; A61N 1/3606; A61N 1/3615; A61N 1/36171; A61N 1/36178

(57) ABSTRACT

A method of identifying a patient with obstructive sleep apnea, implanting in the patient a phrenic nerve stimulator or connecting the phrenic, and adjusting stimulation energy applied by the phrenic nerve simulator to a phrenic nerve in a sleeping patient based on airway obstruction in an airway the patient.

### CROSS-REFERENCE TO RELATED APPLICATIONS

This application claims priority to U.S. Provisional Application No. 63/426,072, filed Nov. 17, 2022, and U.S. Provisional Application No. 63/442,331, filed Jan. 31, 2023, the entire contents of each being hereby incorporated by reference.

### TECHNICAL FIELD

The invention relates to implantable devices to stimulate phrenic nerves to treat airway collapse in patients with Obstructive Sleep Apnea (OSA). The invention may be embodied to exploit a pharyngeal mechanoreflex to stiffen the airway or reverse collapse of an airway, improve gas exchange, and/or enhance sleep quality. The invention is intended to keep a sleeping patient comfortable and asleep while stimulating the phrenic nerve(s) and triggering a reflex to open an obstructed airway in the breathing passage of the patient.

### BACKGROUND

Healthy Sleep is an important part of our lives. It improves physical and mental health. Sleep happens in stages, including REM sleep and non-REM sleep. When humans sleep, their body has a chance to rest and restore energy. A good night's sleep can help us cope with stress, solve problems, or recover from illness. Not getting enough sleep can lead to many health concerns, affecting how we think and feel.

During sleep, a person usually passes through four sleep stages: non-REM N1, N2, and N3, and REM (rapid eye movement). These stages of sleep progress in a cycle from N1 to REM sleep, then the cycle starts over again with N1 or N2. Healthy children and adults spend almost 50 percent of their total sleep time in N2 sleep, about 20 percent in REM sleep, and the remaining 30 percent in the other stages.

During N1, which is light sleep, we drift in and out of sleep and can be awoken easily. Our eyes move very slowly, and muscle activity slows. People awakened from N1 sleep often remember fragmented visual images.

When we enter N2 sleep, our eye movements stop and our brain waves (fluctuations of electrical activity that can be measured by EEG electrodes) become slower, with occasional bursts of rapid waves called sleep spindles.

EEG stands for electroencephalogram. A sleep EEG is a recording of the electrical activity of the brain while you are awake and then asleep. It involves having small electrodes which record the brain activity attached to the scalp.

In N3, extremely slow brain waves called delta waves begin to appear, interspersed with smaller, faster waves until delta waves occur almost exclusively. It is very difficult to wake someone during N3, which is also called deep or slow wave sleep.

When we switch into REM sleep, our breathing becomes more rapid, irregular, and shallow, our eyes jerk rapidly in various directions, and our limb muscles become temporarily paralyzed during sleep. Our heart rate increases, our blood pressure rises. When people wake up during REM sleep, they often describe dreams.

The first REM sleep period usually occurs about 70 to 90 minutes after we fall asleep. A complete sleep cycle takes 90 to 110 minutes on average. The first sleep cycles each night contain relatively short REM periods and long periods of deep sleep. As the night progresses, REM sleep periods increase in length while deep sleep decreases. By morning, healthy people spend nearly all their sleep time in stages 1, 2, and REM.

Although neurophysiology of sleep is not completely understood, it is indisputable that a good night of sleep is a night of continuous, uninterrupted sleep that cycles through sleep stages, including REM, uninterrupted. Sleep disorders, such as OSA, frequently interrupt and disrupt these continuous sleep pattern and lead to daytime sleepiness, fatigue and have many other serious deleterious effects on both mental and physical health.

Obstructive Sleep Apnea (OSA) is a well-recognized dangerous disease that affects millions of people. It can be construed as a sleep disorder that leads to periodic interruptions of lung ventilation that further disrupt sleep.

Pathogenesis of Upper Airway (UA) obstruction during sleep is due to (a) a primary sleep-related loss of UA neuromotor tone and (b) a secondary a lack of adequate compensatory reflex responses that mitigate the obstruction.

In healthy individuals, upper airway stability during sleep is ensured by coordinated and synchronized central control of about 20 (twenty) airway dilator and constrictor muscles (collectively “airway muscles”). The central neural system (CNS) pattern generator (respiratory center) in the medulla of the brain receives inputs from physiologic sensors (also called receptors) via various afferent sensory nerve fibers and controls airway muscles via efferent motor fibers. These physiologic sensors provide physiologic feedback used by the medulla to trigger a reflex in a closed loop reflex arrangement. These reflexes are known as “autonomic” since they do not depend on consciousness. In some cases, the reflexes become insufficient for optimal health. The inventors believe that Obstructive Sleep Apnea (OSA) may be caused by a lack of, or an insufficient, reflex response to an obstructed airway.

Sensory inputs to the respiratory center include signals from chemoreceptors that react to oxygen (O₂) and carbon dioxide (CO₂) in the arterial blood and many distributed mechanoreceptors including ones that react to transmural pressure across the airway wall. In patients with Central Sleep Apnea (CSA) the former “neurochemical” control loop becomes deranged and may be hyperactive. In patients with snoring and OSA the later “neuromuscular” control loop may become insufficiently active to maintain airway patency.

The airway muscles that keep the upper airway open are accessory muscles of respiration that maintain pharyngeal patency during tidal inspiration. Basal tone in these muscles generally declines at sleep onset. The loss of tone makes the airway prone to collapse and obstruct airflow during sleep.

Afferent receptors in the tracheobronchial tree and lungs detect alterations in airway pressure, temperature, air flow, and lung stretch which may be indicators of a collapsed airway. The afferent receptors provide feedback signals to the spinal cord or CNS which may respond to the feedback signals by triggering reflex responses that stimulate the upper airway muscles, which can then mitigate airway obstruction.

Prior investigators suggested that patients with obstructive sleep apnea rely heavily on the aforementioned reflexes to maintain upper airway patency during wakefulness, and that the loss or decrease of reflex activation of the airway muscles during inspiration leads to increases in airway collapsibility during sleep.

Over time, in chronic OSA patients, afferent receptors may gradually desensitize. The patient's brain may fail to adjust to the gradual development of airflow obstruction. OSA may occur because the brain is not receiving adequate signals from the afferent receptors indicating airway blockage. Under these circumstances, airway neuromuscular activity no longer compensates for obstructions in the airway occurring during sleep.

Current evidence indicates that neuromuscular responses in the upper airway musculature must be coordinated with inspiratory activation of the diaphragm and respiratory pump muscles to maintain patency during sleep.

Existing neuromodulation therapies address airway collapsibility by selectively increasing neural signals in the selected efferent branches of the Hypoglossal Nerve (HGN). These branches control protrusion of the tongue by the Genioglossus Muscle (GGM). There is also experimental work directed to selectively increasing other efferent motor control signals to various dilator muscles including the ansa cervicalis that can be instrumental in stiffening of the airway.

Increasing lung volume, especially during expiration, in OSA patients can improve airway patency during sleep. In U.S. Pat. No. 7,970,475 to Tehrani “Device and method for biasing lung volume”, devices and methods are described for increasing lung volume by electrically stimulating the phrenic nerve. This and other prior art teach that stimulation of phrenic nerve is applied to create mechanical traction on the airway, which stiffens the airway and expands the lung to create additional lung volume. One concern of this approach is that it requires high stimulation energy level to treat moderate to severe sleep apnea that may wake patients and are not tolerated by patients.

### SUMMARY

An inventive method and system have been developed and are disclosed here for stimulation of peripheral nerves involved in respiration to take advantage of existing physiologic autonomic control reflex loops. By artificially triggering or otherwise augmenting a physiologic autonomic control reflex loop(s), the method augments and restores natural control of the airway stability and treats OSA by using reflexes to open a closed airway during sleep.

In one embodiment, the method augments the afferent limb of a pharyngeal mechanoreflex, for example the Negative Pressure Reflex (NPR), that naturally dilates and stabilizes the airway in response to increased negative transmural pressure in the airway. NPR is described in scientific literature, and it is recognized that diminution of this reflex during sleep contributes to snoring and airway collapse in at least some OSA patients.

In healthy people during wakefulness, pharyngeal patency is protected by dilator muscles, with negative airway pressure (collapsing pressure) acting as a local stimulus for their graded activation. The respiratory pump can be modelled as a bellow or a pneumatic cylinder where the rapid descent of the diaphragm creates an inrush of fresh air through the nose and down the airway into the lung. This airflow creates significant pressure gradient along the airway that escalates with the increase of the upstream resistance. Since the airway is a collapsible tube, force exerted by this negative pressure during inspiration needs to be opposed to prevent collapse. This opposition is the primary role of the NPR.

NPR manifests by robust and very rapid (within 30-50 milliseconds) activation of pharyngeal dilator muscles when a rapid pulse of suction (negative) pressure is applied by inspiration of ambient air through the nose. Such activation is presumably a protective reflex that allows the pharynx to resist closure during a potentially collapsing perturbation under conditions of increased ventilatory drive while sniffing, exercising, or gasping for air while counteracting challenges of anatomy such as excessive body weight.

During sleep, the afferent neural feedback through the NPR can be used to evoke a coordinated response in multiple accessory muscles that maintain pharyngeal patency without arousing the patient from sleep.

While conducting phrenic nerve stimulation experiments to manipulate lung volume in sleeping patients, inventors realized that these concepts involving reflexes can be exploited for therapy and implemented in embedded software algorithms using known or co-developed device hardware and implantation procedure.

The approach to intentionally triggering NPR to treat OSA disclosed here is counterintuitive and goes against some entrenched beliefs and clinical practices. First, negative airway pressure causes the airway to collapse and the approach of stimulating the phrenic nerve will potentially further collapse the airway by increasing negative pressure in the airway. It is counterintuitive to increase negative pressure to open an airway. Second, clinical practice of phrenic nerve stimulation in individuals with central neurologic disease such as congenital hypoventilation frequently required tracheostomy to prevent airway collapse induced by augmented negative pressure. Third, when a healthy individual is placed in a negative pressure ventilator, e.g., an iron lung, their normal respiratory effort and central chemoreflex frequently cause a reduction or elimination of ventilatory drive. It was observed that in individuals with OSA, the use of negative pressure ventilation increased collapsibility of the airway. Fourth, OSA treatments act to prevent obstruction in an airway. An obstruction is needed, or at least believed needed, to trigger NPR. It is counterintuitive to treat OSA in a way that may allow the airway to be blocked, albeit briefly.

Breaking with tradition and prevailing concepts, the inventors propose to exploit negative pressure conditions in an airway to trigger NPR to treat airway collapse. While observing sleeping OSA patients being treated by phrenic nerve stimulation, the inventors observed that, to their surprise, certain patterns of phrenic nerve stimulation triggered a neural feedback from mechanosensory inputs that opened the collapsed airway almost instantly while acting against the closed upstream airway and against the collapsing negative pressure without waking patient up and disrupting continuous sleep cycle. It was surprising that vigorous diaphragmatic contraction, in response to phrenic nerve stimulation, applied over relatively short periods, was effective in treating OSA when applied during the late expiration and early inspiration phase of the natural breath when the airway is perceived as more collapsible and vulnerable. The substantial abrupt negative pressure was evidenced by the reduction of chest circumference while the abdomen was expanded during the inspiration pattern known as paradoxical breathing.

In one proposed embodiment, the invention augments and restores the NPR in OSA patients during sleep by periodically stimulating one or both phrenic nerves and generating vigorous relatively short contractions (for example, less or equal to 50% of duration of the patient's natural breath) of the diaphragm that generally often coincide with specific parts of the respiratory cycle and more specifically with the late expiration-early inspiration period. In their experiments, inventors observed that such pulses of negative pressure can augment and restore airway patency in sleeping patients with severe sleep apnea and attributed it to reflex activation. The spike of negative pressure generated by the vigorously descending diaphragm, when it occurs in the setting of an occluded or resistive airway, may result in the augmentation of afferent signals from pressure receptors located in the pharyngeal mucosa. These afferent signals are known to conduct information to the respiratory control center of the brain independent of the phrenic nerve via afferent fibers of pharyngeal nerves, such as likely the superior laryngeal nerve from mechanoreceptors in the laryngeal structures and via glossopharyngeal nerve from pharyngeal mucosa.

This nerve firing augmentation may increase afferent signal above the threshold that forces the respiratory central control center to generate efferent signals to various groups of dilator muscles sufficient to stiffen the airway and restore airflow. In this context, if stimulation bursts occur frequently, for example at a natural respiratory rate of 6 to 20 per minute, the airway does not stay closed long enough to impede ventilation or gas exchange in any significant way and oxygen saturation is maintained. It is possible and may be desirable to synchronize the diaphragmatic contraction to the patient-initiated inspiration or to set the physiologically acceptable rate and allow patient to synchronize to the stimulation. In some embodiments only every second or other ratio of breaths are stimulated.

In natural physiology, lung inflation inhibits inspiration. Phrenic nerve stimulation can increase relative inspiratory time by overriding central control. Stimulating the phrenic nerve during expiration brakes exhalation, leading to dynamic lung hyperinflation. As lung volume increases, it exerts caudal traction on the upper airway structures and stiffens the pharynx.

In another embodiment of the invention, phrenic nerve stimulation (PNS) is used to bias or offset the diaphragm or more generally, to brake expiration, producing moderate dynamic lung hyperinflation. This modality of stimulation may be especially efficacious in patients with reduced lung volume. It is well accepted that increased lung volume during exhalation phase of the breathing cycle exerts mechanical caudal traction on the airway. In patients with reduced lung volume, such as owing to significant abdominal visceral fat, restoring lung volume may contribute to airway patency.

Sleep-induced decrements in lung volume can lead to important reductions in longitudinal traction on the airway, yielding an increasingly collapsible pharynx even in the patients with normal lung volume while awake. Some individuals may be quite dependent on this mechanism to maintain airway patency while awake and lose it during sleep. Lung volume biasing may be combined with periodic contractions of the diaphragm, evoking the NPR in some patients.

The lung volume can be increased “statically” by biasing of the lung with the application of constant low-level tone to the phrenic nerve, which prevents complete lung deflation, and exerts caudal traction and stiffens the pharynx.

The lung volume can also be increased dynamically by “expiratory breaking” by increased frequency of phrenic nerve busts or increased inspiratory to expiratory (I:E) ratio, which traps air “dynamically” and prevents complete lung deflation to exert caudal traction and stiffen the pharynx. Increases in the severity of upper airway obstruction will further impede exhalation, and increase the degree of airway trapping and dynamic hyperinflation.

Obstructive sleep apnea (OSA) is the intermittent cessation of breathing during sleep due to the collapse of the pharyngeal airway. Once the airway collapses completely, without intervention, in a sleeping person the airway typically stays collapsed until the patient wakes up (is aroused) by air hunger. This restoration of airways typically takes long enough to cause significant intermittent and periodic oxygen desaturation that has grave consequences for patient's health. This delay is inherent to the physiology since it takes blood some time to travel from the lung to the chemosensors in the brain. In sicker people, such as cardiac patients, this delay may be especially long.

The pharynx (also called the pharyngeal airway or for simplicity just the “airway”) is a tube that connects nasal and oral cavities to the larynx and the esophagus. The pharynx is separated into nasopharynx, oropharynx, and laryngopharynx. The pharynx is a muscular that is collapsible at any point along its passage. There are 20 or more muscles surrounding the passage in the pharynx. These muscles actively constrict and expand the upper respiratory tract lumen. These muscles also contribute to the stiffness of the airway, defined as its ability to withstand negative transmural pressure regardless of its caliber. Stiffening of the airway by mechanical or neural intervention in the context of this patent is called airway stabilization.

Airway muscles can be divided into four groups: muscles that regulate the soft palate position (ala nasi, tensor palatini, levator palatini); tongue (genioglossus, geniohyoid, hyoglossus, styloglossus); hyoid complex (hyoglossus, genioglossus, digastric, geniohyoid, sternohyoid); and posterolateral pharyngeal walls (palatoglossus) pharyngeal constrictors)

These muscle groups interact in a complex way to keep the airway open or closed. Soft tissue structures that form the walls of the upper airway and tonsils include: soft palate, uvula, tongue, and lateral pharyngeal walls.

The site of the airway collapse is significant in the pathophysiology of OSA and in targeting any therapy to prevent collapse. Airway collapse sites that are commonly identified in literature are associated with: Retrolingual space (tongue base), Velopharyngeal space (Soft palate occlusion) and/or Hypopharyngeal space (lateral airway wall occlusion). The Velum (soft palate), Oropharynx, Tongue base and Epiglottis (VOTE) classification on drug-induced sleep endoscopy (DISE) is used widely for classification of collapse cites for obstructive sleep apnea (OSA) syndrome.

FIG. 1 Illustrates the balance of forces that keep airway open during inspiration. Inspiratory negative pressure and extraluminal positive pressure tend to promote pharyngeal collapse. Upper airway dilator muscles and increased lung volume (as it fills with air) tend to maintain pharyngeal patency. Patient 1 inhales air at the atmospheric pressure through the nostrils. Inhaled air travels down the pharyngeal airway 2. Soft palate 8 (sometimes called velum) defines the velopharynx or velopharyngeal space 9 that is the most common location of the airway collapse.

Variables tending to promote pharyngeal collapse include negative pressure 3 within the airway resulting from inspiratory effort and positive pressure 4 outside of the airway. The positive pressure 4 is the product of pressure caused by posture and gravity, fat deposition and other anatomic factors such as small mandible 6. The sum of these pressures 4 and 3 defines the transmural pressure sensed by mechanoreceptors in the airway. Negative inspiratory pressure 3 is dynamic and present during inspiration at any point along the airway. It is proportional to airflow and upstream resistance but increases at any level of ventilatory drive whenever the upper airway obstructs. Conversely, patency is preserved by activation of pharyngeal dilator muscles 5 (e.g., genioglossus and other muscles known but not represented on the FIG. 1) and by increases in lung volume 7, which tends to keep the airway open by longitudinal traction. As a result, dilating forces (e.g., muscle activation) have a complex interaction with collapsing forces generated by anatomy and airway negative pressure.

FIGS. 2A and 2B illustrate reflex control of the airway. The central neural system (CNS) pattern generator (respiratory center) 10 is in the medulla 16 of the brain. The rhythmicity center of the medulla in the brainstem controls automatic breathing during sleep and consists of interacting neurons that fire either during inspiration (I neurons) or expiration (E neurons). I neurons stimulate neurons that innervate respiratory muscles (to bring about inspiration). E neurons inhibit I neurons (to ‘shut down’ the I neurons & bring about expiration). The apneustic center (located in the pons) stimulates I neurons (to promote inspiration). Pneumotaxic center (also located in the pons) inhibits apneustic center and inhibits inspiration. This inhibition can be overcome by phrenic nerve stimulation that affects the respiratory pump directly.

The respiratory center 10 receives inputs from physiologic sensors 11 via various afferent sensory nerve fibers and maintains a patent airway through stiffening and dilation by synchronized contraction and relaxation of muscles via efferent motor fibers. An important airway dilator muscle is genioglossus 14 that protrudes and retracts the tongue. Genioglossus has a direct effect on the velopharyngeal space 9 where airway occlusion often occurs. Such physiologic feedback arrangement is known as a closed loop reflex. Generally, such reflexes are known as “autonomic” since they do not depend on consciousness.

The negative Pressure Reflex (NPR) may be one example of a pharyngeal mechanoreflex activating dilator muscles. A mechanoreflex is a reflex triggered by stimulation of a mechanoreceptor. A muscle spindle stretch receptor, a pressure receptor, a sheer stress receptor or flow receptor can be an example of a mechanoreceptor that reacts to mechanical perturbation, such as deformation and generates afferent neural signal consisting of a train of action potentials in a bundle of nerve fibers.

The NPR is an important physiologic reflex that is evoked and exploited during the proposed therapy. It manifests naturally during every breath by robust and very rapid (within 30-50 milliseconds) activation of pharyngeal dilator muscles. It can also be elicited by a rapid pulse of suction (negative) pressure that is applied through the nose and sensed by transmural pressure sensors in the pharyngeal mucosa. It can be enhanced or induced by electric stimulation of phrenic nerves that causes diaphragmic contraction. The magnitude of the signal sensed by sensors 11 is proportionate to the intensity of diaphragmic contraction and the degree of airway obstruction, particularly in the velopharyngeal space 9. If the airway is occluded (e.g., fully, partially, the like), the pressure becomes more negative and the afferent limb feedback from the reflex into the CNS becomes much stronger. The response of the CNS center 10 is in turn proportionate to the input from the afferent limb 12. This response generates stronger output in the efferent limb 13 which results in the stronger contraction of the dilator muscles 14. Ultimately the entire closed loop response becomes strong enough to open the airway and allow air in. Airway opening, in turn, leads to the reduction of negative pressure and the sensed signal in the afferent limb 12. The closed loop system comes into steady state and respiratory stability can be restored.

FIGS. 1, 2A and 2B are simplified to illustrate the main elements of pharyngeal anatomy and innervation. Because of the physiological importance of maintaining pharyngeal patency and the many tasks required of this portion of the airway (speech, swallowing, etc.), a sophisticated motor control system has evolved, with about 20 upper airway muscles playing a part. The following paragraphs expand the complexity of this natural arrangement for maintaining the airway open and prior attempts to improve it in OSA patients.

During natural inspiration, negative intra-luminal pressure pulls three soft tissue elements, the tongue, posterior pharyngeal walls, and soft palate, toward each other, thereby reducing the airway lumen in the velopharyngeal region. This airway-collapsing action is opposed by pharyngeal dilator muscles, including the genioglossus, geniohyoid, and tensor and levator veli palatini. Additionally, activation of the pharyngeal constrictors stiffens the airway walls.

This choreographed natural activation of pharyngeal muscles maintains airway open in wakefulness but often fails in sleep state. One purpose of the described invention is to evoke, potentiate and exploit this process of natural airway tightening by the NPR when the natural excitatory traffic to corresponding motor-neurons is insufficient to keep the airway open during sleep. This approach is novel since NPR, while known since 1980s, has never been proposed as a therapy. It is advantageous over prior art, since prior art proposed to control only specific efferent muscle motoneurons (rather than sensory afferents) to evoke individual, uncoordinated response from pharyngeal dilator muscles.

The soft palate (the velum) comprises muscle and tissue, which makes it mobile and flexible. When a person swallows, the soft palate rises to seal the opening of the airways to prevent pressure from escaping through the nose. The shape, position, and movements of the soft palate are maintained by five pairs of muscles, including tensor veli palatini (TVP), levator veli palatini (LVP), palatopharyngeus (PP), palatoglossus (PG), and musculus uvula (MU). The tensor veli palatini muscle (tensor palati or tensor muscle of the velum palatinum) is a broad, thin, ribbon-like muscle in the head that tenses the soft palate.

The tensor veli palatini is supplied by the medial pterygoid nerve, a branch of mandibular nerve, the third branch of the trigeminal nerve—the only muscle of the palate not innervated by the pharyngeal plexus, which is formed by the vagal and glossopharyngeal nerves. The tensor veli palatini tenses the soft palate and by doing so, assists the levator veli palatini in elevating the palate to occlude and prevent entry of food into the nasopharynx during swallowing.

The palatoglossus muscle functions as an antagonist to the levator veli palatini muscle. Palatoglossus arises from the palatine aponeurosis of the soft palate, where it is continuous with the muscle of the opposite side, and passing downward, forward, and lateral in front of the palatine tonsil, is inserted into the side of the tongue, some of its fibers spreading over the dorsum, and others passing deeply into the substance of the organ to intermingle with the transverse muscle of tongue. It is innervated via vagus nerve (via pharyngeal branch to pharyngeal plexus). It elevates posterior tongue, closes the oropharyngeal isthmus, and aids initiation of swallowing. This muscle also prevents the spill of saliva from vestibule into the oropharynx by maintaining the palatoglossal arch.

The genioglossus muscle (GGM) receives input from the brainstem respiratory central pattern generator via the Hypoglossal Nerve (HGN). The presence of ‘pre-activation’ (hypoglossal nerve firing 50-100 ms prior to the phrenic nerve) supports the presence of pre-motor inputs to the hypoglossal motor nucleus in the medulla.

The function of GGM in health and disease is extensively studied and described in literature. For example: Cori J M et. al., Sleeping tongue: current perspectives of genioglossus control in healthy individuals and patients with obstructive sleep apnea. Nat Sci Sleep. 2018 Jun. 15; 10:169-179.

The hypoglossal nerve, also known as the twelfth cranial nerve, cranial nerve XII, or simply CN XII innervates the GGM and was the foundation for the first successful neuromodulation technique to treat OSA. HGN stimulation to treat OSA was disclosed in U.S. Pat. Nos. 5,158,080 and 5,540,733.

While successful in some, HGN stimulation is not an effective solution for many patients. In some cases, effectiveness could be restored by increasing the power applied to the nerve, but many patients cannot tolerate the increased power regimen for one reason or another. A possible reason for this is that the acceptable level of GGM activity is not sufficient to overcome other physiological changes that occur and persist during sleep, such as low activity of the other dilator muscles, altered co-activation patterns with the other dilator muscles and low lung volumes that results in the reduced caudal traction of the airway. These limitations are addressed in this application through novel approaches such as manipulation of lung volume and transmural airway pressure via stimulation of phrenic nerve.

FIG. 3 further illustrates the role of NPR in the pathogenesis of OSA. In healthy persons and unhealthy OSA patients during wakefulness, pharyngeal patency 21 is maintained by the phasic activation of pharyngeal dilator muscles 20, with negative airway pressure (collapsing pressure) acting as a local stimulus to their activation. The negative pressure reflex is a protective reflex that allows the pharynx to resist closure during a collapsing perturbation. The dilator muscles respond within tens of milliseconds to negative pharyngeal pressure, thereby maintaining airway patency.

To overcome compromised pharyngeal anatomy 22, such as in common obesity, suboptimal tongue anatomy, or mandible anatomy etc., the upper airway dilator muscles of a patient with OSA must be more active during wakefulness than those of healthy individuals. In wakefulness NPR responds to the increased (more negative) negative pressure in patients with compromised anatomy. The sensed response is a product of the smaller pharyngeal lumen and the need for greater intrapharyngeal pressure to generate adequate airflow. This increased negative pressure drives greater pharyngeal dilator muscle activation. Thus, the airway muscles compensate for the deficient anatomy of the OSA patient while awake, and the ventilation is maintained. Even in patients with very severe OSA, disordered breathing events occur only during sleep, emphasizing the importance of central control in the pathogenesis of this disorder.

It is known that neuromuscular reflexes are reduced 24 during sleep 23. The ability of the pharyngeal dilator muscles to respond to negative pressure is substantially attenuated during sleep even in healthy people. Loss of these excitatory inputs to the efferent hypoglossal motoneurons may greatly decrease the ability of the genioglossus and other upper airway dilator muscles to respond to negative pressure 25 compared to wakefulness. Loss or reduction of this reflex mechanism during sleep would be expected to precipitate large decrements in muscle activity and subsequent airway closure 26. As a result, if an individual's pharyngeal anatomy is compromised, their airway will be unprotected by NPR and vulnerable to collapse during sleep. In OSA, airway closure leads to hypoxia and hypercapnia 27, which evokes a CNS chemoreflex. Unlike mechano-reflexes such as NPR, chemoreflexes depend on the blood circulation for response and may take as long as 15 to 90 seconds to produce the response from the respiratory pump 28 and increased respiratory effort. These delays manifest as periodic breathing and apnea hyperpnea cycles. Ultimately increased respiratory effort is often accompanied by arousal 29 and restoration of wake level of activity of pharyngeal dilators 20. As the cycle can repeat itself as frequently as 20 to 90 times an hour, the patient's sleep is compromised.

The invention may be embodied as a method of treating obstructive sleep apnea (OSA) by periodically stimulating at least one phrenic nerve of a patient where the stimulation is applied while the pharyngeal airway of the patient is naturally obstructed.

The invention may be embodied as a method of treating OSA by periodically stimulating at least one phrenic nerve of a patient where stimulation is applied while the airway is closed (e.g., fully occluded) or partially occluded (e.g., occluded sufficiently to cause, for example, blood oxygen desaturation).

The invention may be embodied as a method of treating OSA by periodically stimulating at least one phrenic nerve of a patient where stimulation is applied while the airway is characterized by increased obstruction.

The invention may be embodied as a method of treating OSA by periodically stimulating at least one phrenic nerve of a patient where stimulation is artificially applied while the airway is closed.

The invention may be embodied as a method of treating OSA by periodically stimulating at least one phrenic nerve of a patient where stimulation is applied while the airway is closed or partially occluded where the stimulation is applied with the energy sufficient to generate diaphragmic muscle contraction sufficient to generate negative airway pressure sufficient to trigger the NPR, which opens the airway.

The stimulation may include a stimulation burst initiated while the airway is closed. A substantial proportion, e.g., greater than 50%, 75% or 85%, of the stimulation bursts may be simulation bursts initiated when the airway is closed. The stimulation burst(s) may be applied first at a first energy level sufficient to generate action potentials in phrenic nerve and later at second energy level sufficient to evoke reflex opening of the collapsed airway by activation of upper airway muscles. The stimulation burst(s) may be applied first at a first energy level sufficient to generate action potentials in phrenic nerve and further at a second energy level sufficient to evoke reflex opening of the collapsed airway by potentiation of a mechanoreflex. The mechanoreflex may be a negative pressure reflex.

### BRIEF DESCRIPTION OF THE FIGURES

FIG. 1 is a cross-sectional view of an upper portion of an airway passage in a patient.

FIGS. 2A and 2B show a patient's head, brain, and upper airway to illustrate reflex control of an airway in the patient.

FIG. 3 is a flow chart showing the connection between airway stability and negative pressure reflex (NPR).

FIG. 4 is a flow chart showing restoration of pharyngeal muscle tone by phrenic nerve stimulation that evokes NPR.

FIG. 5 is a cross-sectional view of a patient with a phrenic nerve system including an implanted electrode and an implanted pulse generator.

FIG. 6 are charts showing variations over time of airway flow, respiratory flow, oxygen level and electrical simulation current applied to phrenic nerve.

FIG. 7 are charts showing variations over time of air passage flow rate and respiratory effort during a stimulated breath.

FIG. 8 is a flow chart for adjusting a parameter(s) for simulation of the phrenic nerve to treat sleep apnea.

FIG. 9 is a chart illustrating an energy titration curve comparing degree of diaphragm contraction with energy (current in mA) applied by an electrode to a phrenic nerve.

FIG. 10A is a flow chart of an algorithm for the detection of the capture threshold.

FIG. 10B is a flow chart for an algorithm for the detection of the therapeutic threshold.

FIG. 11A is a graph illustrating examples of ramping stimulation energy during therapy titration to determine optimal stimulation energy followed by applying the optimal stimulation energy during a night of therapy. FIG. 11B is a graph that illustrates different parameters that may be set according to certain example embodiments.

FIG. 12 shows patient in bed during sleep therapy, wherein the patient has an electrode stimulating a phrenic nerve, an implanted pulse generator, and a bedside computer monitor that is wirelessly communicating with the implanted pulse generator and communicating with a cloud computer.

FIG. 13 schematically illustrates an implantable pulse generator that stimulates the phrenic nerve to treat OSA.

FIG. 14 is a block diagram of electronic components of an implanted pulse generator.

FIG. 15A is a flow chart of an algorithm to optimize use of negative pressure reflex in the sleeping or resting patient.

FIG. 15B is a flow chart of an algorithm to increase lung volume to treat OSA.

FIGS. 16A and 16B are charts illustrating adjusting stimulation rate optimize phase locking of breathing using the phase angle between stimulation pulse trains and spontaneous breathing effort of the patient.

FIGS. 17A and 17B are charts illustrating use of lung volume to optimize and improve effectiveness of phrenic nerve stimulation to treat OSA.

FIGS. 18A and 18B are charts illustrating spectral power analysis.

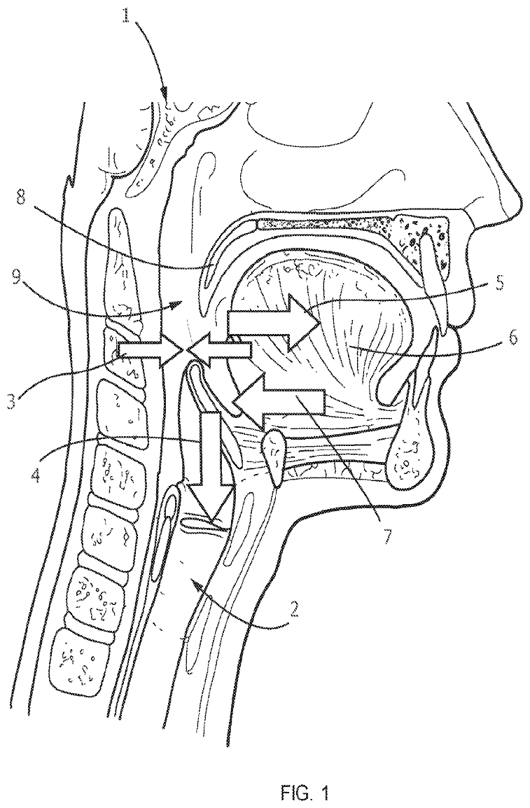

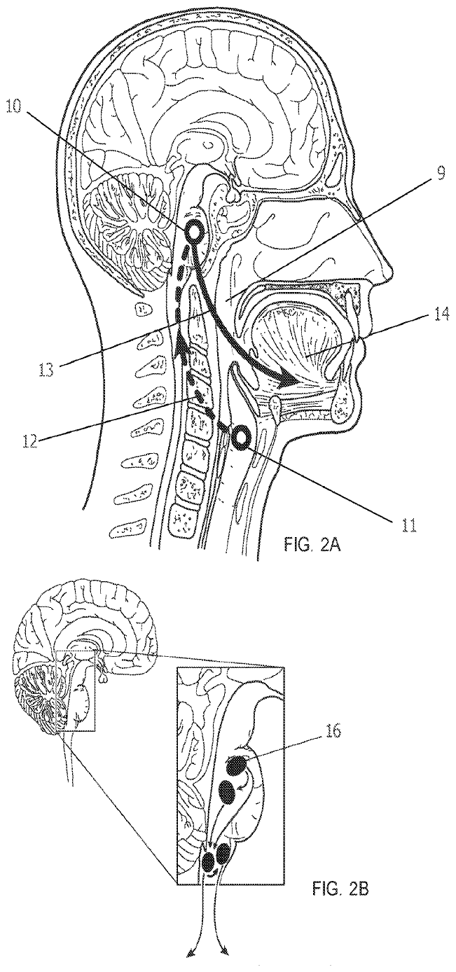

### DETAILED DESCRIPTION OF INVENTION

Sections are used in this Detailed Description solely in order to orient the reader as to the general subject matter of each section; as will be seen below, the description of many features spans multiple sections, and headings should not be read as affecting the meaning of the description included in any section.

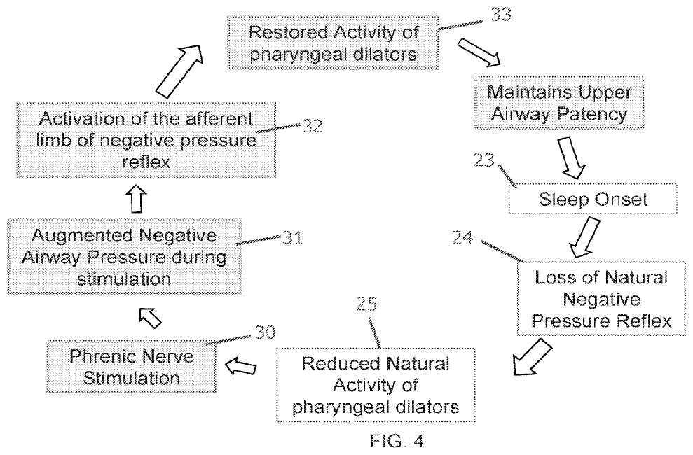

### Description of FIG. 4

FIG. 4 illustrates the restoration of pharyngeal muscle tone by phrenic nerve stimulation that evokes NPR. This is a foundation of the proposed therapy. Sleep onset 23 inevitably leads to reduction of natural NPR 24. The reduced reflex leads to the reduction of naturally occurring periodic efferent limb signaling to muscles responsible for maintaining airway patency 25. This results in greatly increased inspiratory airway resistance and likely intermittent airway collapse.

The inventors developed a therapy that includes periodic stimulation of phrenic nerve 30 that results in a robust (e.g., greater than 200 ms, 250 ms, or 300 ms train length) and vigorous diaphragmatic contraction (e.g., greater than 100 uA above the diaphragmatic twitch capture threshold as discussed elsewhere herein). The diaphragmic contraction immediately (e.g., within tens of milliseconds) generates negative pressure 31 within the airway. This pressure change is picked up by pressure sensors in the pharyngeal mucosa and the afferent limb of the NPR is potentiated 32. This leads to the reflex activation of the efferent limb and contraction of dilators, including but not limited to genioglossus 33, which restores airway patency. Since NPR is very fast, this process can be cyclically repeated at a rate consistent with natural breathing (6 to 20/min). As a result of this periodic activity, the airway never stays closed long enough to induce hypoxia and activate respiratory chemoreflex. It naturally takes an OSA patient tens of seconds of apnea to drop past a standardized desaturation threshold (e.g., 3-4% oxygen hemoglobin saturation from a baseline), which is considered clinically significant. When certain example techniques discussed herein are applied to every beat, or every second breath, the oscillatory cycle of apnea-hyperpnea does not occur or is greatly attenuated and sleep disruption is prevented.

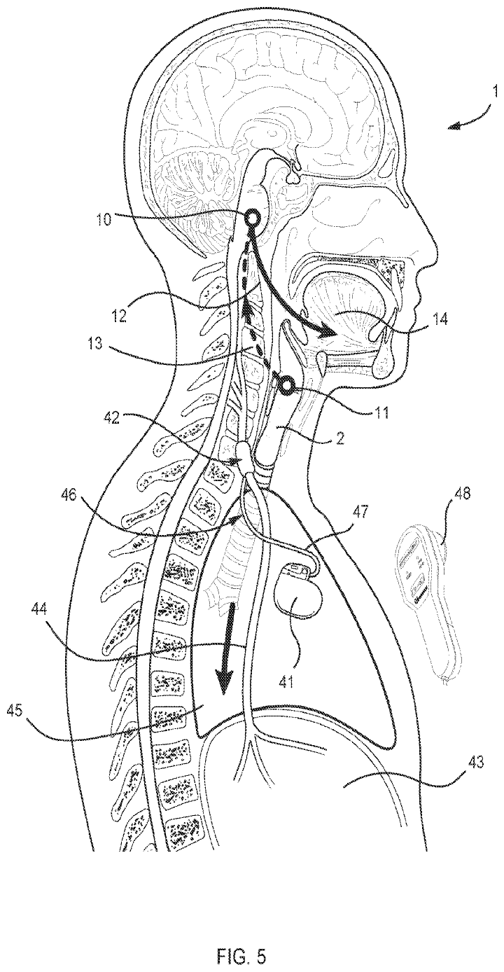

### Description of FIG. 5: Phrenic Nerve Stimulation System

FIG. 5 schematically illustrates one physical embodiment of the invention. Patient 1 is implanted with a nerve stimulation system 46 including an implantable pulse generator (IPG) 41 that is electrically connected to the electrode system 42 that is implanted in proximity to the phrenic nerve 44. A stimulation burst from the IPG generates vigorous contraction and descent of the diaphragm 43. The contraction of the diaphragm fills the lung 45 with air and generates negative pressure in the airway 2, which may be closed. Negative pressure is sensed by the receptor 11 that activates the afferent limb of the NPR. Respiratory center 10 responds by generating efferent signal 12 that activated the dilator muscles illustrated by genioglossus 14. It is understood that other dilator muscles are also co-activated in the natural physiologic sequence. The airway is dilated and stiffened by synchronized effort of muscle groups activated by the reflex. The negative pressure is strongest if the airway is occluded and that facilitates restoration of airway patency.

Phrenic nerve stimulation to assist or replace natural breathing is known in the art of implantable and partially implantable nerve stimulators. As illustrated by the FIG. 5, a phrenic nerve stimulation system 46 can include an electrode sub-system 42 adapted to apply electric current to one or two of the phrenic nerves 44 (e.g., the left and/or right phrenic nerves) in a pattern. The pattern can be programmed into and/or embedded into memory (such as microprocessor memory) that is included in IPG 41. The pattern may be predefined and/or dynamically determined based on one or more characteristics of the patient. In some examples, the pattern for the electric current that is supplied by the IPG is controlled by an external computing device (e.g., wand 48) that communicates (via wired or wireless communication) to the IPG to cause the electric current to be supplied to the phrenic nerve(s) 44 in accordance with a pattern that is controlled by the external computing device. Stimulation can be monopolar, bipolar, or multipolar and apply energy to either or both right and left phrenic nerves.

The phrenic nerve stimulation system 46 further includes a lead 47 electrically connecting the IPG 41 and the electrode 42. In certain example embodiments, the phrenic nerve stimulation system 46 may also include sensors and/or processing for detection of respiration states (inspiration, expiration), sensing of airflow, chest motion, and/or pressure.

Example implantable devices and systems suitable for phrenic nerve stimulation are available from Integer Holdings Corporation.

The electrode sub-system 42 may be a nerve cuff, an endovascular electrode, a paddle electrode, or a percutaneously inserted tubular electrode lead approximating phrenic nerve in the neck or in the chest. It may be connected to the IPG or a subcutaneous wireless antenna in communication with an External Pulse Generator (EPG)—not shown—(e.g., that is placed outside the body of the patient) by a flexible lead. It will be appreciated the techniques discussed herein in connection with IPG 41 may be similarly applied in connection with an EPG.

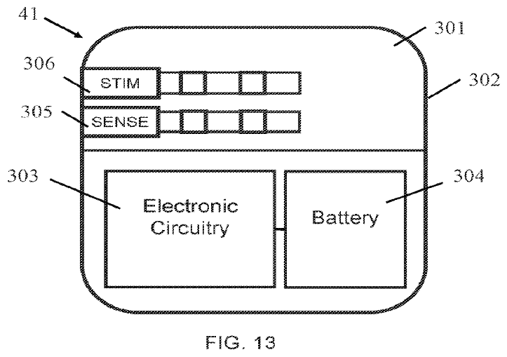

The IPG 41 can include an implanted battery. The battery may be rechargeable or single use. In some examples, energy can be transmitted wirelessly by a transdermal RF link from an external device outside of the body of the patient. In some examples, the IPG 41 can be equipped to provide telemetry such as via a Bluetooth™ transceiver. Additional details of example implementations for an IPG are discussed below in connection with FIGS. 13 and 14, for example.

In some examples, the IPG/EPG component of the phrenic nerve stimulation system 46 includes any or all of: a hardware processor (e.g., a microprocessor, a transceiver, memory (e.g., flash RAM, cache, volatile memory, non-volatile memory, read-only memory, etc.) associated circuitry, and embedded software (which may include and/or be firmware in certain examples) that is configured to be executed by the hardware processor of the IPG/EPG to perform the operations defined in the instructions. Such instructions may include instructions required for activation (e.g., of the IPG/EPG) and deactivation (e.g., of the IPG/EPG) by physician, patient, or other user. In certain examples, one or more computing devices may provide a user interface (e.g., a graphical user interface) for adjusting stimulation parameters that include current, voltage, pulse duration, and frequency (e.g., pulse frequency, which, as discussed below, may be between 20 and 50 Hz, for example), pulse burst rate, duty cycle, and a burst shape parameter. The values for such stimulation parameters may be stored in the IPG 41. Illustrative values for these stimulation parameters are discussed below.

The wireless communication to the IPG 41 can be performed using a handheld computer device 48 (e.g., a “wand”) that is configured to modify stimulation parameters, the embedded software of the IPG, and upload/download data to/from the IPG when brought within close distance with the IPG that is implanted within the patient's body. The computer device 48 may include a display and user input keys to allow a user, e.g., physician (or the patient or other user in certain examples), to view data collected from the IPG and change operating parameters such as the stimulation parameters—e.g., rate and energy level and the like.

Phrenic nerve stimulation (PNS) can improve airway patency through the physiologic mechanism of activation of a mechanoreflex such as the NPR and by the increase of lung volume. PNS can be achieved through the use of a hardware system, such as the Phrenic Nerve Stimulation System 46 that is described in connection with FIG. 5. The PNS can be embedded in such a hardware system. One of the fundamental issues of any nerve stimulation therapy, such as PNS, is the compromise between effectiveness and the ability of the patient to tolerate therapy. The effectiveness of therapy is generally proportional to electric field energy applied to the nerve by the IPG. The IPG generates electric current pulses that cause the generation of action potentials in the targeted nerve fibers (e.g., motor fiber in the phrenic nerve bundle) that innervate targeted muscle fibers. Often untargeted nerve fibers are also activated limiting patient's tolerability. The tolerability may include many factors such as pain, muscle twitching, unpleasant sensations and interference with respiratory mechanics, gas exchange and sleep quality. Accordingly, the embedded software in the IPG (or other components of an example hardware system) can include features to titrate energy to thereby achieve a compromise between effectiveness and tolerability.

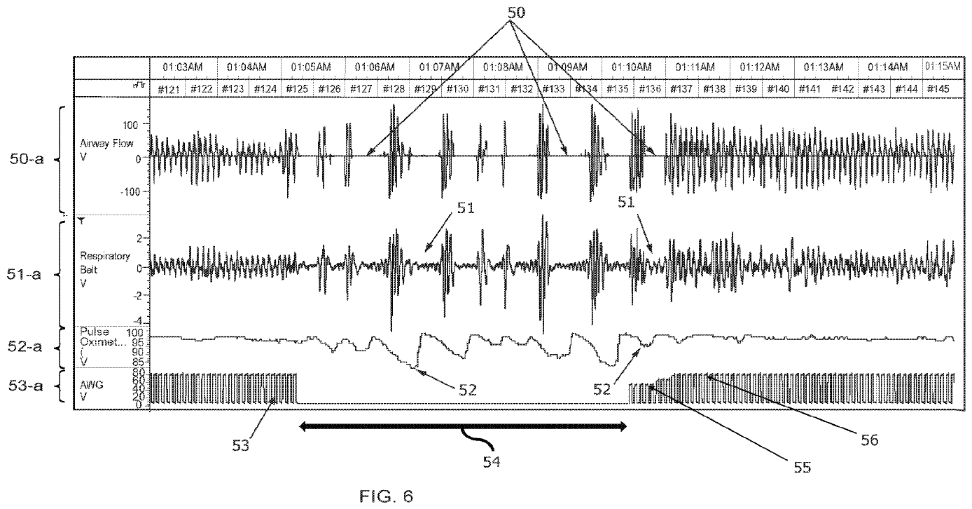

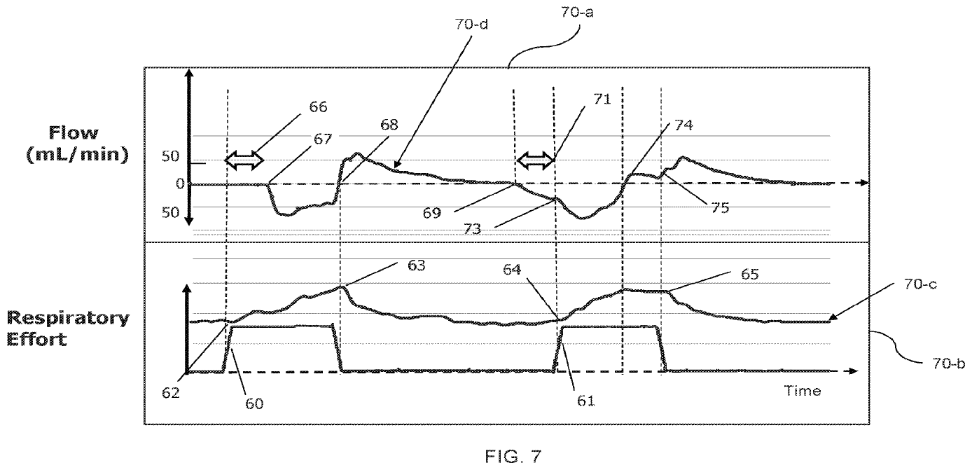

### Description of FIGS. 6-7: Illustrative Clinical Data

Data for the graphs shown in FIGS. 6 and 7 was obtained based on a patient that was wearing a nasal mask attached to a precision air flow meter. The patient was equipped with thoracic and abdominal respiratory belts, finger pulse oximeter, and standard polysomnography (PSG) montage as commonly used during sleep studies. Percutaneous electrode was inserted in their neck close to the left phrenic nerve and connected to a bedside electric pulse generator operating in constant current mode. Stimulation parameters that included bipolar pulse trains of 150 microseconds long square pulses were applied at the current of 1 to 5 mA, at 30 Hz. Adjustments are made by the operator to the stimulation current in 0.25 mA increments to achieve the desired stabilization of breathing.

The patient was instrumented with a standard PSG montage during the experiment that included EEG and EMS electrodes. According to sleep stage analysis during and after the experiment the patient was sleeping throughout the experiment (in Non-REM sleep). There were observed microarousals that terminated apneas. The fact that the patient slides right back into apneas instantly when the stimulation is turned off supports the concept that upper airway patency and ventilation can be stabilized during sleep without waking patients up, and that PNS can decrease AHI, desaturations, clinically significant arousals, and sleep fragmentation, all of which should alleviate OSA clinical symptoms (hypersomnolence, fatigue, etc.).

FIG. 6 shows charts presenting therapy results in connection with application of artificial nerve stimulation over a period of sleep with the illustrated therapy performed by inventors for a patient with very severe OSA. During the control time period 54 phrenic nerve stimulation is turned off. During the control period 54, the patient immediately experienced severe OSA, as evidenced by the absence of airflow (first trace from the top showing airflow sensor signals 50-a) during apnea periods 50, presence of respiratory effort 51 (second trace from the top showing respiratory belt sensor signals 51-a) during apnea, and oxygen desaturations 52 (third trace from the top showing pulse oximeter measurements 52-a). The abruptness of the transition to OSA is one clear indication that the patient was asleep before and after the transition. OSA is a condition associated with sleep. Patients generally don't fall asleep and develop severe OSA in an instant.

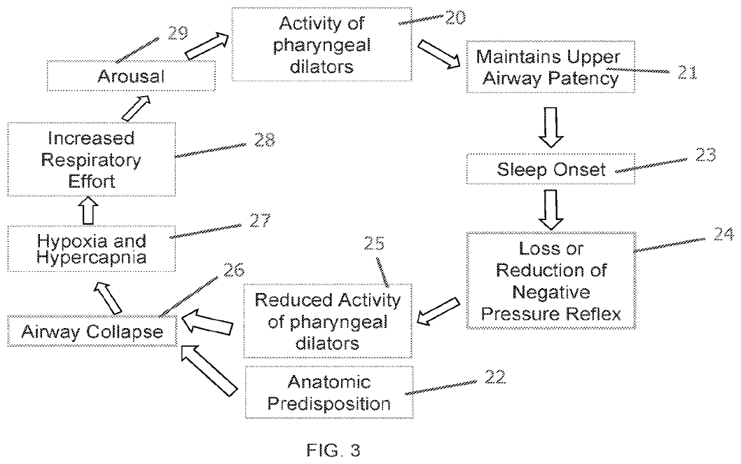

Oxygen desaturation periods 52 follow apnea periods 50 after a circulatory delay. Oxygen desaturation and accompanying rise of CO₂ enables chemoreflex to arouse the patient (which may include causing the patient to wake up) and terminate the apnea period by restoring airway patency. This is the cycle naturally occurring during OSA and illustrated by FIG. 3. When stimulation is turned on during periods 53, 55, and 56, the patient's breathing is vastly improved. Stimulation bursts of sufficient magnitude (e.g., in accordance with settings for one or more of the stimulation parameters) evoke reflexes that likely include the NPR and open the airway almost instantly without awakening the patient as illustrated by FIG. 4. Blood gases O₂ and CO₂ are maintained, and patient does not experience significant periodic breathing, sleep disruptions or long periods of apnea.

Element 53-a shows the output from an arbitrary waveform generator (AWG) that can be used in connection with controlling or delivering the stimulation energy to the patient. The AWG can be used to define the current shaping waveform and can be used to, for example, map an analog voltage to current with a bipolar constant current stimulator. In certain examples, a rise/fall and peak amplitude is assigned to the waveform to be generated to supply a physiologically relevant diaphragm tetanizing pull, where the rise is designed to ensure a diaphragm pull that is fast enough to trigger the NPR of the patient (e.g., between about 50 ms to 500 ms), but not so fast that it causes arousal from the rapid tetany (e.g., between about Oms and 50 ms). In certain examples, and as noted elsewhere herein, within that shaped waveform is a plurality of biphasic pulses that may be between about 20-50 hz. The amplitude of those pulses can be set to match wherever they land in time on the current control carrier waveform.

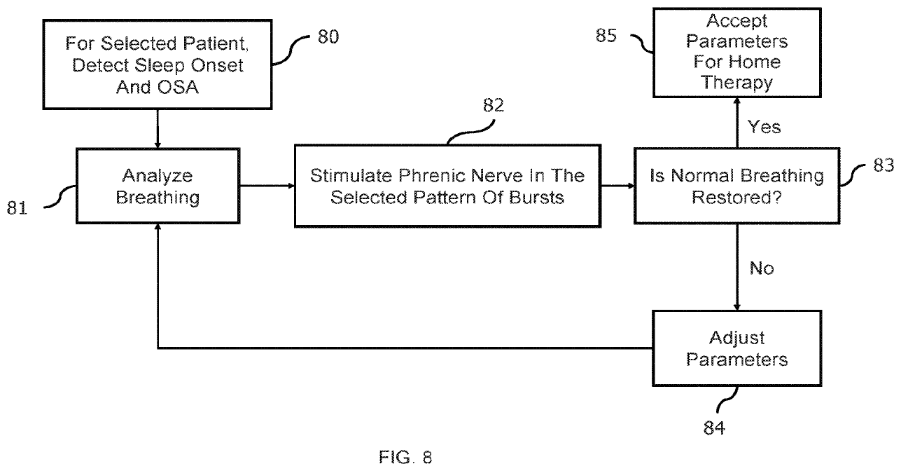

increasing the energy level of phrenic nerve stimulation from level 55 to level 56 resulted in gradual more complete resolution of airway obstruction. During relatively low level 55 of phrenic nerve, the patient continued to experience airway occlusions as evidenced by the periods of no airflow in airflow at 50, as well as the corresponding episodes of low or no respiration effort in at 51 and drops in blood oxygen levels in at 52. Increasing the stimulation level from the level at 55 to the level 56 stops airway occlusions (returns to normal breathing) as evidenced by the airflow shown in airflow signal 50-a, the respiration effort shown in 51-a, and the higher and/or more consistent levels of blood oxygen levels in 52-a (all of which occur during stimulation level 56). This process is further illustrated by FIG. 8.

Return to normal breathing occurs abruptly as the phrenic nerve stimulation level exceeds an unknown threshold. The abruptness is shown by the periods of no airflow stopping and immediately followed by a relatively continuous level of strong airflow. The abrupt transition indicates that a reflex is triggered when the stimulation level exceeds the threshold, wherein the reflex may be the negative pressure reflex. When triggered, a reflex will cause the muscles in the airway to open the airway.

FIG. 7 includes an airflow rate chart 70-a and respiratory effort over time chart 70-b. The charts illustrate data that correspond to two breaths from the same patient during a portion of the therapy period illustrated in FIG. 6. Stimulation bursts 60, 61, e.g., called pulse trains, are applied at a respiratory rate that approximates the patient's natural breathing rate (e.g., between about 6 and 20 BPM). The stimulation bursts in this case have a duration approximately equal to ⅓ of the breath (e.g., a duty cycle of 33% or an IE Ratio of 1:3). In certain examples, I:E ratios from about 4:1 to 1:4 can be used to trigger the reflex. In some examples, an inverse (e.g., inspiration longer than expiration and equivalent I:E ratios (1:1)) can be beneficial when utilized (e.g., in oxygen therapy) in critical care mechanical ventilation. In some examples, the longer an “I” time holds the distal airways open the more time is available for perfusion resulting in improved washing out of CO₂. This reduces the hypoxic burden, normalizing a person's respiration/perfusion outside of stimulus. The first stimulation burst 60 is initiated when the patient's airway is closed as evidenced by air flow signal 70-d being zero during period 66, despite the onset of inspiratory effort. The respiratory effort signal 70-c represents abdominal girth measured with a respiratory belt, which increases at the onset of PNS (e.g., below 50 ml is severe flow limitation/apnea, between about a 50 and a 150 mL lung volume increase corresponds to a mild flow limitation, while greater than about 150 mL in lung volume corresponds to a patent airway without such a flow limitation), and increases even further when the airway opens and that lungs inflate. An airflow of zero with respiratory effort present indicates that the airway is occluded, the patient is indeed asleep, and their base pharyngeal muscle tone is not sufficient to keep their airway open. This observed airway collapse (evidenced by no airflow during respiratory effort) is further evidence that the patient is asleep since the OSA is a “sleep induced” condition.

As shown in FIG. 7, the airway opens abruptly, and inspiratory airflow starts 67 after a time delay 66. This time delay is the time it took the respiratory effort of the diaphragm, natural and/or stimulated, and negative pharyngeal pressure to reach the afferent signal threshold that activated the reflex opening. Patient then inspires at the peak airflow rate of >50 ml/min, which indicates unobstructed airway. The bottom trace illustrates the respiratory effort signal 70-c (e.g., the measured abdominal girth—circumference that has been calibrated based on or to lung volume (e.g., in mL)) that is indicative of inspiratory effort (diaphragmic excursion). The respiratory effort signal 70-c in this example has been calibrated to lung volume (mL). Beginning of effort 62 coincides with the stimulation 60 but precedes inspiratory airflow 67 by the delay 66 indicating diaphragmic motion and negative airway pressure buildup against the closed airway. Inspiration stops and turns into expiration at the point 63 when the central control initiates the exhalation phase of breath and phrenic nerve stimulation is turned off.

As shown in FIG. 7, the stimulation 60 of the phrenic nerve sufficient to trigger the negative pressure reflex (see level 56 in FIG. 6) allows the patient's natural negative pressure reflex to quickly open 67 a closed airway to allow the patient to inhale during the inspiration cycle. Without the stimulation 60, the airway may have remained closed during the inspiration cycle, and may have remained closed for two, three, or more inspiration cycles until the patient awakes and gasps for air. The stimulation 60 triggers the negative pressure reflex to allow the patient to breath without the patient suffering no airflow during multiple consecutive inspiration cycles and without being awakened.

The next breath is initiated by the respiratory center of the patient. The airway is obstructed but not closed, as evidenced by airflow 69. The airflow is limited by airway resistance and peaks at approximately 10-30 ml/min (e.g., a range in which an airway flow limitation can occur); however subsequent stimulated breaths can then rise to approximately 30-60 ml/min peak flow, which can resolve the flow limitation (e.g., completely) without waking the patient (e.g., below an arousal threshold). It will be appreciated, there is a threshold to the stimulus that may be delivered. For example, if the stimulation is too high, then the patient may be aroused (e.g., by hitting overstretch receptors, which can trigger the pain center) and/or wake up.

Inflection point 73 coincides with the onset of the second stimulation burst 61 after the delay time 71. Airflow is accelerated and abdominal excursion indicates significant diaphragmic contraction (effort). Inspiration is terminated by the respiratory center at the point 74 where airflow is reversed and becomes exhalation at a modest rate. The continuation of the PNS burst into the expiratory phase decreased exhalation flow rates to a modest level until the inflection point 75 where expiratory flow accelerates and returns to normal level 75 similar to one without PNS. It coincides with the termination of the stimulation burst 61 and cessation of effort 65. In certain examples, respiratory effort can be tied to, based on, or derived from tidal volume from the patient. In terms of mL volume, approximately 50 ml-150 ml can be considered a mild flow limitation, greater than approximately 150 ml can be considered normal patient airway without limitation, and less than about 50 ml being a severe flow limitation (e.g., an apnea).

### Description of FIG. 8: Therapy Selection Algorithm

FIG. 8 illustrates an example algorithm of therapy selection. A patient can be identified to have moderate or severe OSA based on standard home PSG test. For example, the patient may have apnea hypopnea index (AHI)>20 events per hour. Patient is implanted with an IPG (e.g., such as described herein, for example, in connection FIG. 5, 13, or 14) and a phrenic nerve stimulation electrode. IPG is confirmed operational (e.g., confirming that the IPG can stimulate the phrenic nerve to cause diaphragm contraction in the press parameter range), and patient is discharged for a period needed to heal, such as one month. In some examples, the patient is brought to the office of the sleep physician specialist for therapy activation. In some examples, this step can be also performed in the home setting using a remote sleep monitoring device and a telemedicine session.

While patient is sleeping (80) their breathing pattern and sleep pattern are analyzed by standard instrumentation or custom instrumentation used for sleep studies 81. Stimulation of phrenic nerve is initiated using an IPG that is controlled or set to an initial set of parameters 82 (which is an example the stimulation parameters discussed herein). In some examples, values for the initial set of parameters can include setting a rate (e.g., the pulse burst rate) to a number close to the patient's natural breathing or a different reasonable rate comfortable for the patient. A Duty cycle (burst duration) parameter can be set to Inspiration:Expiration or (I:E) ratio of (1:3), (1:1), (1:2) or another suitable initial number, and stimulation current is gradually increased until diaphragmic contractions corresponding to the stimulation bursts are detected (e.g., clearly).

At 83, the process determines if normal breathing has been restored for the patient based on the stimulation of the phrenic nerve from 82. In other words, the process determines if the patient's OSA has been resolved due to stimulation of phrenic nerve. For example, the process may determine that the AHI of the patient has been reduced by at least 50%. If normal breathing has been restored, then the patient may be set to continue therapy at home 85 with the selected set of parameters (e.g., based on the initial set of parameters or those that have been modified per 84 discussed below) and instructions to initiate therapy every night.

If, however, normal breathing of the patient has not been restored, the parameters can be changed at 84. The change in values for the parameters can include changing one or more of the multiple parameters that are used to control stimulation of the phrenic nerve. For example, the stimulation parameters can be changed and titrated upwards, toward more stimulation power, energy, or intensity, until OSA is resolved.

As an illustrative example, stimulation current (e.g., which can be representative of, or related to, energy delivered to the nerve) can be increased. Such increased current generally results in stronger diaphragmic contractions until muscle fibers are fused and the muscle cannot contract more.

Another example of adjusting values of the stimulation parameters may include changing the rate at which amplitude of pulses in the burst is increased, often called ramp time. This can be shortened to generate more vigorous abrupt diaphragmic contractions.

Another example may include controlling a duty cycle parameter and/or stimulation rate (e.g., to be increased with the understanding that some air trapping may occur during stimulation if stimulation bursts are more frequent or last longer). Duty cycle burst duration/breath duration may be, or correspond to, the I:E ratio discussed herein. However, in certain examples, duty cycle may be expressed as percentage of breath (e.g., total breath). Accordingly, for example, 30% may correspond or mean that inspiration is 0.3 of total breath. Some patients may benefit from a lung volume increase during sleep to prevent lung collapse and loss of caudal traction exerted by the lung inflation on the airway. All stimulation parameters are titrated based on patient's tolerance. It is anticipated that after patient adapted to therapy, the intensity of stimulation may be increased.

### Titration and Auto-Titration of Therapy

Patient tolerability to phrenic nerve stimulation is not a constant but a function of environmental factors and neural plasticity. Changes that occur over time are often referred to as the nervous system's adaptation to stimulation. In the case of phrenic nerve stimulation therapy is applied primarily during sleep. During sleep the brain can adapt to rhythmic sensations such as intended or unintended proprioceptive inputs, muscle motion, or even tingling. People sleep well on fast moving trains and aboard swaying ships, for example, after they adapt to it.

After implantation of electrodes some time needs to be allocated to allow to heal and inflammation at the surgical site to subside. This can be a 30-day period, for example. After that, therapy can be activated, and a period of adaptation and adjustment can begin. This process can be completely implemented by the caregiver, by the patient, or at least partially automated. When therapy is applied during sleep at home, some automation can be beneficial.

Activation of therapy can include ramping up stimulation energy until the first induced diaphragmic contraction is detected (motor neuron capture). Capture is often described as a muscle “twitch.” Energy can be increased further until the full engagement of all nerve fibers in the nerve bundle and muscle fibers in the muscle after which no more muscle contraction results from the further increase of stimulation energy (fused or tetanized muscle).

While the gold standard for measuring strength diaphragmatic contraction (also called “respiratory effort”—e.g., as shown in FIG. 7) is an esophageal balloon pressure, other methods such as respiratory belts, bioimpedance, magnetometry, accelerometry, and inspiratory pressure measurement can be used as a dependent variable in the creation of a therapy titration curve. These surrogate variables are intended to characterize or approximate respiratory effort that results from muscle contraction. Bioimpedance (e.g., electric tissue impedance) in the context of this patent is the response of a living organism to an externally applied electric current. It is a method for estimating body composition, in particular air contained in the lungs and airways, where a weak electric current flows through the body and the voltage is measured in order to calculate impedance (e.g., resistance) of the body tissues that are part of the closed electric circuit and are in the current return path. Since breathing changes air volume in the lung, impedance can be used to track breathing.

The general need for up-titration of any nerve stimulation therapy is well recognized. For example, U.S. Pat. No. 11,529,514 describes up-titration of Hypoglossal Nerve (HGN) Stimulation treatment for OSA. In case of the HGN the protruder muscle is activated by stimulation and airway obstruction by tongue and soft palate is moved in some cases. The so-called velopharyngeal space may be seen at a valve or a trapdoor that opens or closes the pharyngeal airway in response to tongue protrusion. Phrenic Nerve (PN) is different from HGN in that it does not innervate pharyngeal muscles but instead activated muscles of the respiratory pump. In the context of the proposed therapy, and compared to more traditional HGN stimulation, phenic nerve stimulation, activation and titration of therapy is complicated by the need to evoke negative pressure reflex and airway traction, while maintaining blood gases and lung volume that are critical for the overall functioning of the human body. This is further complicated by a desire to accomplish such responses while keeping patient comfortable and asleep.

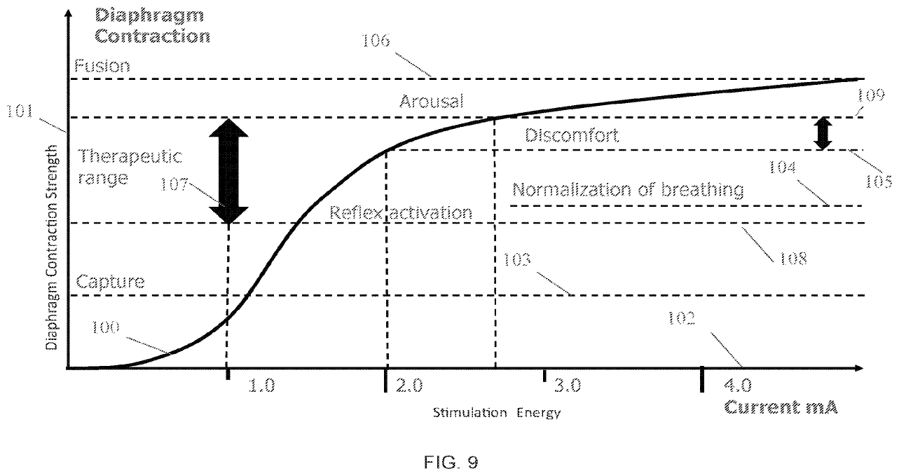

### Description of FIG. 9

FIG. 9 illustrates an energy titration curve 100 that may be first created during therapy activation and adjusted at any time after, on a schedule or as needed. The curve can be generated as an automated experiment and stored in the non-transitory memory of the IPG—e.g., as a table or an equation. It can be uploaded into the cloud computer storage, become part of patient therapy history, and made available in a graphic or reduced to a numeric form to a physician and to the patient to evaluate, monitor, and guide therapy.

Curve 100 can be plotted as a relation between stimulation energy 102 (horizonal axis) and the diaphragm contraction strength 101 (vertical axis). In certain example embodiments, the diaphragm contraction strength may be represented as an index that is based on, for example and as discussed below, integrated diaphragmic EMG, ultrasound imaging, esophageal pressure, accelerometry, exhaled airflow, lung volume, thoracic bioimpedance, and/or airway pressure. Stimulation energy in this context is different from the traditional electric engineering definition. Nerve conduction is not strictly an electric conduction in the traditional sense. Nerves are stimulated to generate or “fire” a series of action potentials. Different fibers in a nerve bundle have different activation energy thresholds. In more general terms, the more fibers in the phrenic nerve that are firing and faster they are firing action potentials, the stronger the diaphragmic contraction and the respiratory effort will be. This effort becomes negative pressure in large airways, and if the airway is open, the pressure gradient generates air flow. The airflow over time becomes “breathing” or volume exchange, which is also called tidal volume, for an individual breath and minute ventilation for the volume inspired and expired over a minute.

In popular constant current systems, stimulation energy is usually expressed as stimulation current in mA with the voltage and pulse duration kept constant. The so-called constant voltage systems may also be used in connection with the nerve stimulation techniques discussed herein- and may be interchangeable with the necessary technical adjustments. In terms of nerve firing, response current and pulse duration (e.g., the length of individual pulses within a given burst) maintain an inverse relationship. Another way to adjust stimulation energy is changing the voltage differential between the cathode and the anode applied by constant voltage systems where the nerve interface impedance is considered relatively stable.

In the case of a phrenic nerve, the stimulation pulse train frequency is generally maintained between 20 and 40 Hz and individual pulse durations between 50 and 250 microseconds. Diaphragmic contraction strength cannot be easily measured but it can be approximated and/or indexed based on an integrated diaphragmic EMG, ultrasound imaging, esophageal pressure, accelerometry, exhaled airflow, lung volume, thoracic bioimpedance, and/or airway pressure as expanded later herein.

Generally, the IPG is set to deliver energy in the operating range confined between the lowest (or low) capture level 103 and highest (or high) muscle tetanic contraction level 106 when all the muscle fibers in the diaphragm are fused and the muscle cannot contract anymore. Therapeutic range 107 is defined as the range of energy delivery where the clinically significant improvement of airway resistance can be expected. The upper level of the therapeutic range 107 is always above capture limit 103 and below the tetanic contraction level 106.

There is a safety margin between levels 105 and 106 dictated by patient's comfort, diaphragmic muscle fatigue, or blood gas exchange which can be impeded by overinflation of the lung. This safety margin is reflected in two tolerance levels 105 (the arousal limit) and 109 (the sensation tolerance limit).

Sleep, Arousals from Sleep, and Awakening

Sleep dysregulation and sleep disorders are associated with cardiovascular, metabolic, and psychiatric disorders. Sleep dysfunction is usually evaluated in sleep clinics, through analysis of nocturnal polysomnography (PSG). A PSG recording involves measuring electroencephalography (EEG), electrooculography (EOG), electromyography (EMG), electrocardiogramairflow, respiratory effort, and blood oxygen saturation during a night's sleep. PSGs are conducted at night, and result in the scoring of Sleep Disordered Breathing (SDB) events. Those are the number of apneas or hypopneas per hour of sleep called Apnea Hypopnea Index (AHI), number of periodic leg movement (PLM) events per hour of sleep with and without associated arousals, and sleep stages. Sleep stages are wakefulness (W), non-REM sleep (stage one N1, two N2, or three N3) or REM sleep, reported as percent of total sleep time. AHI can be reported either as “recommended” by the American Academy of Sleep Medicine (AASM), which includes only hypopneas associated with 4% oxygen desaturation; or the AASM “alternate” AHI which counts apneas associated with 3% oxygen desaturation and/or arousal. A typical sleep study also reports sleep latency, latency for sleep onset to the first epoch of REM sleep, and sleep efficiency (SE), the percent of time asleep when in bed. A slight variation of sleep efficiency is Wake After Sleep Onset (WASO), which, unlike SE, only considers wake after sleep onset has occurred. In the context of sleep stage scoring, sleep stages are attributed to successive 30 second epochs, arbitrary practice attributed to the historic use of paper printing in sleep studies.

In sleep medicine “arousals” don't necessarily mean waking up but can mean shifting to a lighter sleep stage. In the context of this application clinically significant arousals are best defined according to “The AASM Manual for the Scoring of Sleep and Associated Events” current at the time of writing. In certain example embodiments, awakenings are defined as any sleep stage shift to wakefulness for more than 15 seconds. As of today, the gold standard for detecting arousals is through visual inspection of PSG recordings. Accepted practice and current standards distinguish microarousals (3-15 seconds) and wake (>15 seconds), a distinction which is also arguably arbitrary.

Microarousals can occur naturally as part of normal sleep-wake physiology. as a result of external stimuli such as PN or HGN stimulation, or internal sleep disorder events such as SDB (e.g., sleep apnea). In general, microarousals are not considered clinically significant events unless they result in sleep stage disruption (e.g., if a patient ends up not feeling rested, sleepy during the day, or generally indicates a poor night of sleep).

In a simplified, less formal way, arousals can be defined as sleep disruptions that may not wake the patient but are significant enough and frequent enough to prevent deep healthy sleep. OSA events are commonly associated with arousals that occur when the apnea is terminated by hypercapnia or hypoxia. These arousals temporarily restore breathing, generally do not wake patient up, but prevent them from progressing to deeper sleep stages and REM sleep. If phrenic nerve stimulation causes frequent arousals (e.g., that may be significant) coinciding with stimulation bursts, then stimulation energy is likely too high.

Referring to FIG. 9, a patient's sensation tolerance limit 109 can be determined as a level that is based on or at where the conscious patient feels pain or discomfort. Arousal tolerance limit 105 can be determined as a level that is based or at which the sleeping patient manifests frequent clinically significant arousals that may be registered during a home PSG or a sleep lab sleep study. The patient tolerance limits 105 and 109 are generally not a constant but are subject to change because of different external and internal factors including patient's adaptation to therapy.

For an individual patient, arousal limit 105 may be lower or higher than discomfort limit 109 and generally the lower limit determines the maximum acceptable energy for that specific patient during the specific period of time or for a set of health conditions. For example, if a patient gains or loses weight, undergoes surgery, or the like, then their limits may be permanently changed. If a patient has a cold, a flu, or the like then their limits (e.g., including values for other stimulation parameters) may be only fluctuating (e.g., temporarily changed). The stimulation settings can be seen as an electronic prescription under these conditions.

One or more (e.g., Several) settings and/or tolerance levels may be stored in the non-transitory memory of the IPG memory resulting from any (or all) of the different tests discussed herein. It is generally expected that the discomfort tolerance level will increase with accommodation and that higher tolerance results in more effective therapy.

A goal of the PNS therapy to treat OSA is to maximize the effectiveness, such as to minimize AHI, within the patient's effectiveness and tolerance range. In this context, it can be expected that therapeutic effects can only manifest at energy levels above the capture level (also called a “capture threshold”) 103. Above that, there may be other threshold levels that may include: 1) reflex activation level 108 (e.g., where the negative pressure generated by stimulation has measurable/detectable effect on the upper airway airflow limitation); and 2) breathing normalization level 104 (e.g., where apneas and hypopneas are no longer detectable or clinically significant).

For example, clinical data represented by FIG. 6. illustrates how increasing the energy level of phrenic nerve stimulation in steps from level 55 to level 56 in a sleeping patient with OSA resulted in gradually more complete resolution of airway obstruction indicated by airflow.

The therapy titration may be implemented and/or adjusted in a doctor's office during a polysomnography (PSG) study, also sometimes called a sleep study. However, this can lead to frequent patient visits, repeated sleep exams, and can lead to under-delivery of therapy because of the patient's reaction and refusal to accept increased energy levels (or just failure to show up for the test). Accordingly, there is a strong need to automate the process of therapy initiation, dosing, and/or titration based on objective input measurements to optimize therapy effectiveness, reduce involvement of physicians, and to reduce the number of visits to clinics for the IPG programming. It is recognized that a patient's response to therapy may change night to night, during the same night, and with a change of sleep position. Thus, these measurements likely need to be frequently repeated.

In some examples, an IPG may have some bult in sensing capability such as an accelerometer, blood pressure pulse, oximetry, ECG sensing, or bioimpedance. However, it will be appreciated that the precision and/or responsiveness of such systems may not be comparable to home and office PSG systems.

An illustrative example of a device that can provide sleep monitoring is the WatchPAT® Home Sleep Apnea Device (HSAT) that utilizes the peripheral arterial signal for OSA and CSA diagnosis. It measures up to 7 channels including tonometry, heart rate, oximetry, actigraphy, body position, snoring sounds, and chest motion via three points of contact. WatchPAT is commercially available from Zoll-Itamar. The algorithms described here can be implemented using a home monitoring device, similar to the WatchPAT but modified to communicate with the IPG directly or using a suitable wireless interface. The communication device can include an antenna, which is attached to or held close to the patient during the night. Alternatively, a dedicated custom wearable device or system can be developed to monitor patients sleep at home and communicate with the sleep physician, patient, and the IPG as expanded further in this application.

In certain example embodiments, the techniques described herein may be implemented on mobile devices, such as a smart watch or smart phone that may be configured and/or programmed to communicate with an IPG as described herein.

Other example systems for sleep monitoring may also be leveraged. An example of such systems includes inertial systems that are configured to monitor or measure acceleration, position, and/or angle of the chest wall (or other areas of a patient's body). When data from such monitoring is appropriately filtered and integrated, a reliable signal can be produced that may then be leveraged in the IPG design described herein. While this technique may tend to have weaker amplitude during quiet rest breathing, hyperpnea that engages accessory muscles in the chest and neck may be more detectable. Thus, periodic breathing can be detected.

Where the terms “asleep” and “awake” are used the determination can be used using standard PSG methods described elsewhere in this patent, in the current edition of AASM Manual for the Scoring of Sleep and other widely accepted standards. To determine sleep quality and stage FDA approved PSG systems typically rely on multi-electrode EEG (electroencephalogram), EOG (electrooculogram), EMG (electromyogram), ECG (electrocardiogram), pulse oximetry, and other sophisticated measurements. These systems are available for use in a sleep clinic and at home but are not generally suitable for frequent use. They allow strict criteria for neurologic definitions of sleep stage, arousals, AHI, ODI parameters of OSA, and periodic breathing.

It is appreciated that simplified methods for detection of patient's sleep versus awake and OSA versus normal breathing states are also relevant and may be more practical in the night-to-night home setting even if those are less accurate than PSG. For example, if a patient is supine and not moving, then their Heart Rate (HR) is slow, constant, and stable. When their breathing rate (BR) is slow, constant, and stable, the patient is likely to be asleep. In this context, “slow” means a set of individual values for the patient, but generally HR can be expected to be 50-70 beats per minute and BR 6 to 20 breaths per minute. In the patient is experiencing an OSA episode, their breathing, and heart rate are likely to become highly variable in a much broader range. The heart rate may vary between 40 and 120 beats per minute within the time window of 1 or 5 minutes and breathing can change from barely detectable effort to hyperventilation and tachypnea at 20 to 30 breaths per minute. Thus, variance of HR and BR can be used as a criteria of therapy success. The periodic nature (periodicity) of these variations is an indication of OSA pattern. It can be expected that cycles of heart rate and breathing variability repeat every 30 to 120 seconds. This period or periodic breathing frequency is another individual characteristic of the patient that is stored in the system memory. The coherence between periodic signals such as breath volume, BR, HR, pulse pressure and pulse oximetry can be another indication of periodic breathing.

### Phases and Muscle Mechanics of Periodic Breathing

Periodic breathing (PB) is frequently defined as a breathing pattern characterized by crescendo/decrescendo changes in tidal volume and is commonly to be due to systemic mechanisms destabilizing breathing such as heart failure. It is associated with CSA but can be present in OSA also. During the hyperventilation phase of the cycle there is a reduction in CO2. If the CO2 tension decreases below the apneic threshold, a respiratory pause ensues. During the apneic phase of the cycle the CO2 increases and the O2 tension decreases, thus driving subsequent hyperventilation. For the purpose of this patent PB simply means that patients breathing pattern during sleep consists of discernable periods of apnea or hypopnea followed by periods of hyperventilation that perpetuate themselves unless treated or interrupted by awakening. A patient is likely to have a clinical diagnosis of mild or severe OSA, CSA, or Mixed Apnea according to existing AASM guidelines. OSA is by far the most common diagnosis. In such cases, first line therapies such as weight loss, drugs, and CPAP may have failed for the patient.

Hyperventilation is sometimes called over-breathing. Hyperventilation or hyperpnea is breathing in excess of what the body needs. In the case of OSA, bouts of hyperpnea follow prolonged apnea periods and often overcompensate, remove too much CO2 from blood leading to temporarily low respiratory drive that affects both respiratory pump and the airway tone. This phenomenon is part of the pathogenesis of periodic breathing and present in both OSA and CSA phenotypes. Hyperventilation occurs at the end of airway obstruction after the obstruction is resolved by the chemoreflex activated airway opening. Hyperventilation, in contrast to rested breathing, vigorously engages muscles in the upper thorax. This phenomenon can be used to detect OSA using an accelerometer built into the IPG design and implanted in the upper chest over the pectoral muscle or elsewhere under the skin of the chest and over the rib cage.

From a functional point of view, there are three groups of respiratory muscles: the diaphragm, the rib cage muscles and the abdominal muscles. Each group acts on the chest wall and its compartments, e.g., the lung-apposed upper rib cage, the diaphragm-apposed low rib cage and the abdomen. Contraction of the diaphragm expands the abdomen and the lower part of the rib cage (abdominal rib cage). In the restful sleep contraction of the diaphragm is normally all that is required to generate tidal volume and support metabolic needs.

The rib cage muscles, including the intercostals, the parasternal muscles, the scalene and other neck muscles, mostly act on the upper part of the rib cage (pulmonary rib cage) and are both inspiratory and expiratory. The two sternocleidomastoideoles originate from the mastoid process of the temporal bone and the superior nuchal line of the occipital bone. These muscles can elevate the anterior ribs. Therefore, they are used as accessory muscles in pulmonary ventilation.

Activity of neck inspiratory muscles (NIM), particularly the scalene muscles is increased in hyperventilation and can be monitored in order to monitor abnormal breathing. In certain example embodiments, stimulation can be periodically interrupted for one or more breaths (e.g., a couple of breaths such as 2, 3, or 4) to obtain an EMG recording (e.g., that is clean). An acetometer can be integrated into the electrode system to directly monitor muscle contraction. A separate electrode sub-system can be added to the lead electrically isolated from the phrenic nerve stimulation electrode to assess EMG.

The abdominal muscles act on the abdomen and the abdominal rib cage and are expiratory. A highly coordinated recruitment of two or three muscle groups is required to support elevated ventilatory effort in exercise of hyperventilation. During breathing at rest, this is accomplished by the coordinated activity of the diaphragm.

### Feedback, Starting, and Ramping Up Therapy

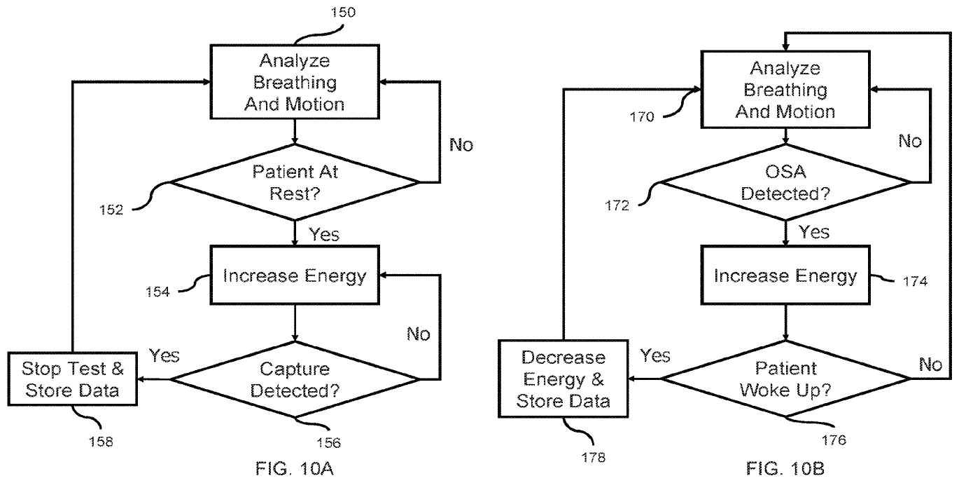

The algorithms (e.g., computer processes) described herein are illustrations and embodiments that can be implemented in software that is stored to non-transitory memory of IPG 41. In some embodiments, algorithms can be distributed between several internal and external devices that are connected by wired or wireless communication links. As an example, some steps or processing may be performed on an external device (e.g., a smart phone, smart watch, etc.) that is in communication (e.g., wirelessly) with an IPG. Two embodiments are illustrated in FIGS. 10A and 10B. The primary function of the IPG is to deliver stimulation pulses to the phrenic nerve of a patient. Control of how stimulation pulses are delivered is performed (at least in part) by computer program code that may be stored in memory. The control may be based on an internal timing that is derived or acquired from a clock (e.g., a real-time clock). Sensing and logic functions can be integrated into the IPG or reside outside of the patient's body (e.g., on another computing device, such as a smart phone or the like).

### Description of FIGS. 10A and 10B

FIG. 10A illustrates a process, which may be embodied as a computer implemented algorithm, for the detection and/or determination of the capture threshold (e.g., 103 as shown in FIG. 9). At a certain time (e.g., when patient is expected to go to bed, is detected as laying down in bed or the patient signals that the patient is starting a sleep cycle), the process starts at 150 and monitoring of the patient is performed. The patient is monitored with, for example, actigraphy and/or accelerometry to confirm that patient is supine and resting at 152. A resting state may include supine or reclined position maintained for some time as well as other parameters such as low motion activity, stable low heart rate, or breathing rate. If the patient is not resting, then the process loops back to 150 and further analysis is performed. If the patient is resting, then the stimulation energy from the IPG is increased at 154. In certain example embodiments, the increase may be controlled via a microprocessor in a low power, hibernation state, or state of monitoring only. In certain examples, the full range of current from the IPG that may be supplied during therapy can be between 0 to 5.0 mA. Stimulation energy can be increased (e.g., in 154) in graded steps of, for example, 0.1 mA to 0.25 mA, between a range of 0.5 mA and 2.5 mA. Based on (or in response to or in conjunction with) an increase in stimulation energy, the process checks, at 156, if the capture threshold has been reached by detecting the first distinct rhythmic twitches of the diaphragm muscle. If the capture threshold is detected, then the values used for the various parameters of the therapy are stored at 158 (and any other data for the performed test) and the test is terminated. If, however, the capture threshold has not been detected, then the process loops back to 154 and the stimulation energy is increased. The process continues until the capture threshold is detected or a threshold amount of stimulation energy is being applied. In certain examples, For example, pulse trains of stimulation pulses 0.5 to 1.5 sec long can be applied every 1 to 3 seconds to create a distinct, easy to detect periodic pattern of contractions.

FIG. 10B illustrates a process, which may be embodied as a computer implemented algorithm, for the detection of a therapeutic threshold, which may include any or all of the thresholds used in connection with therapeutic range 107 and/or thresholds 206 and 207. For the process, at 170, the patient is monitored with PSG or other monitoring techniques, which may be internal to IPG, external to the patient/IPG, or combined, to confirm that patient is supine, resting, and likely sleeping. As part of this monitoring, the process determines if OSA has been detected at 172. Periodic airway occlusion is generally associated with sleep state. If no OSA is detected, the process continues to monitor the patient. If, however, OSA is detected, then the stimulation energy can be increased in graded steps. In certain examples, an initial or first simulation energy setting for the processing performed in FIG. 10B may be the capture threshold that is determined/stored per the processing performed in FIG. 10A. Using the capture threshold, the process can increase the stimulation energy in steps (up to a defined maximum allowed threshold such as 5.0 mA) at 174 in order to determine a therapeutic range that is between a first threshold (e.g., the lowest energy level at which the breathing pattern is altered in systematic periodic way), and a second threshold at which breathing is stabilized (apneas are no longer detected).

The processing may also include determining if the patient has woken up at 176. If the patient has woken up, then the data associated with therapy at this time may be stored at 178 and a further instruction provided to decrease the stimulation energy that is being applied. In certain examples, The waking up threshold for the patient can be stored in memory for future use and periodically updated. Pulse trains of stimulation pulses with the duty cycle of 30 to 70% can be applied at (or as close as possible to) the natural breathing rate of the patient that could be previously detected when at rest but not yet showing the OSA pattern. It is understood that the natural breathing rate has a normal variability typical for living organisms.

An embodiment of the therapy optimization algorithm (e.g., which may be implemented using a microprocessor or the like) can be a gradual periodic stepwise increase of delivered energy to gradually accommodate the patient's sleep needs and utilize brain plasticity to increase patient tolerance. The gradual periodic stepwise increase of delivered energy can be called an “energy ramp”. The energy ramp can be implemented breath to breath, quarter hour to quarter hour, hour to hour, or night to night in small increments. Accordingly, for example, the processing performed at 174 may be confined to adjusting the stimulation energy about every 15 minutes in some cases, and every breath or every other breath in other cases.

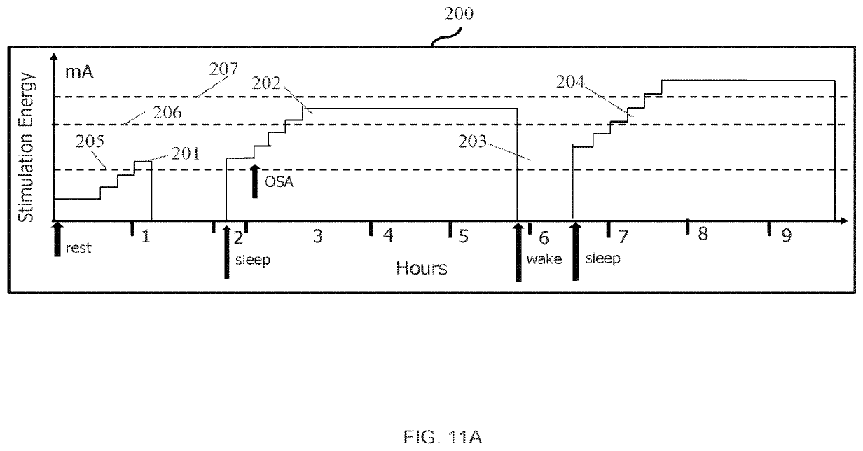

### Description of FIGS. 11A and 11B

In connection with such energy ramps, FIG. 11A illustrates an example of energy ramps serving the purpose of therapy titration and optimization during one night of therapy during and after therapy activation. The individual patient's operating range is determined during the activation visit to the doctor's office of the monitored activation sleep night in a sleep lab or at home (e.g., using the processing shown in FIG. 10A). This can be a PSG or home monitored night, for example, where a patient's sleep quality is monitored by the physician in real time using telemetry.

Referring more specifically to FIG. 11A, a graph 200 is shown that includes, on the x-axis, a length of time in hours. This corresponds to different points in time during the patient's sleep. On the Y-axis is the stimulation energy applied via an IPG to the patient. Initially a test ramp 201 is applied and is used as a range finding ramp. The ramp 201 shown in FIG. 11A is performed after the patient rests in bed but before they are asleep or display OSA. It enables determination of the resting capture threshold 205 (e.g., as described in connection with FIG. 10A). At the same time, other parameters such as resting breathing rate can be detected and stored for later use (e.g., stored to non-transitory memory of a device). The device memory can be included as part of the physical IPG microprocessor, coupled to the IPG microprocessor (e.g., on the same piece of silicon), or in non-transitory memory of an external device that is in wireless communication with the IPG. Data relevant to the individual patient's therapy such as historic and current parameters, settings, preferences, and decisions may also be stored. As used herein, the data relevant to the individual patient's therapy may be called a patient treatment plan.

When the patient falls asleep and manifests OSA, their individual OSA patterns such as time period/frequency of apneas and post-occlusion hyperventilation morphology can be determined and stored in memory for use by future automated therapy. These measurements may be made for the patient sleeping on their back, side, prone, reclined and other sleeping positions of the body and neck. The position detection system can be calibrated at the same time for the future detection needs.

For example, depending on the patient's treatment plan stored in the device memory, after the range finding ramp is completed, stimulation can be turned off or continue at some low level of energy above capture threshold to help patient get used to the rhythmic sensation.

After patient is confirmed to likely be sleeping, stimulation can be increased to a level known to be within the therapeutic energy range from the previous sleep history. The level of energy may be specific to the patient's position since many patients can be expected to sleep on their side, back, or reclined. A patient's position can be determined using inertial sensors such as accelerometry and gyroscopes integrated into the IPG electronics or by an external monitoring device such as a wearable device or a radar-based motion monitoring device. The monitoring device can be a mmWave radar patient monitoring apparatus, a motion detecting camera, or the like. Millimeter wave (mmWave) radars transmit electromagnetic waves and any objects in the path reflect the signals back. By capturing and processing the reflected signals, a radar system can determine the range, velocity, and angle of the objects. The potential of mmWave radar to provide millimeter level precision in object range detection and its indifference to clothing and bed linens makes it a suitable non-contacting technology for sensing human bio-signals during sleep.

Ramp 202 is executed after the patient falls asleep and manifests OSA or other forms of periodic breathing. Periodic breathing can be detected by the sensors and programmed logic included in the IPG or communicated from an external device to the IPG. The ramp is stopped after the first therapeutic threshold 206 is achieved where the periodic breathing is sufficiently reduced or eliminated. For example, calculated AHI or rate of O2 desaturations by more than 3% may be reduced from about 50-120 per hour to about 0-15 per hour for the individual patient as a part of their therapy plan. Reduction of AHI by a given percentage (e.g., greater than a 50 percent reduction, and in certain examples above 90 percent and up to a 100 percent reduction) from a baseline without therapy may also be an individual patient's goal. In certain example embodiments, a percentage of sleep time above 90% O2 saturation and/or other relevant criteria can be used individually or in combination with other elements, such as AHI, to improve therapy for a patient. As discussed previously, alternatives to PSG can be used to determine OSA severity and sleep quality.

During the night, at 203, the patient may wake up, sit up, and get up and walk around. In this case stimulation pulses may be stopped (e.g., with or within express input from the patient) or reduced to a comfortable level depending on the patient's treatment plan. After the patient returns to bed and falls asleep again, a second therapeutic ramp 204 may be executed. In some examples, the re-execution of the ramp (or execution of the second ramp 204) may occur automatically and/or without any express input from the patient. It will be appreciated that the automatic control of the stimulation pulses (including reinitialization of the stimulation pulses) and control of the ramping may be advantageous as individuals will occasionally get up during the night, only to fall back to sleep. The automatic control described herein may thus alleviate a need for a patient to expressly turn on/off the stimulation. Rather, as discussed elsewhere herein, the stimulation may be automatically controlled based on various factors including the position of the individual, their determined sleep stage, and the like.

FIG. 11B is a graph 1100 that illustrates different parameters that may be set according to certain example embodiments. In some examples, the execution (either at the beginning of a individual's sleep for the night or in the middle) of the stimulation may be based on a latency time parameter 1102 (e.g., a therapy onset delay). The latency time parameter may be manually, automatically, otherwise dynamically determined. The latency time parameter may be set to be between about 0 and 60 minutes, with typical values being, in certain examples, between about 30 and 40 minutes. In certain example embodiments, a therapeutic ramp time parameter 1104 (e.g., the onset ramp shown in FIG. 11B) may also be used that can be manually, automatically, otherwise dynamically determined. The value for the therapeutic ramp time can be, for example, between 0 and 60 minutes, and may typically be, in certain examples, about 30 minutes.

For example, a midnight interruption may use a lower value for the latency time parameter than when the individual is initially falling asleep for the night. In some examples, the time for when stimulation is executed, or resumed, may be a function of the value of the latency time parameter in combination with other measured values (e.g., any or all of the measured values from the individual). The combination of these values may be used to calculate when stimulation is to be executed (and/or stopped).

The ramp may be executed until the therapeutic threshold is found that satisfies the preset criteria or a pre-planned maximum tolerance limit is reached. The second therapeutic threshold 207 may be higher or lower than the first therapeutic threshold 206. For example, a patient may have slept on their side during the first ramp and on their back during the second ramp or otherwise changed some positional or physiologic parameters that affect airway collapsibility. Additional parameters may be similarly set in connection with the time for the therapeutic window and a ramp off.

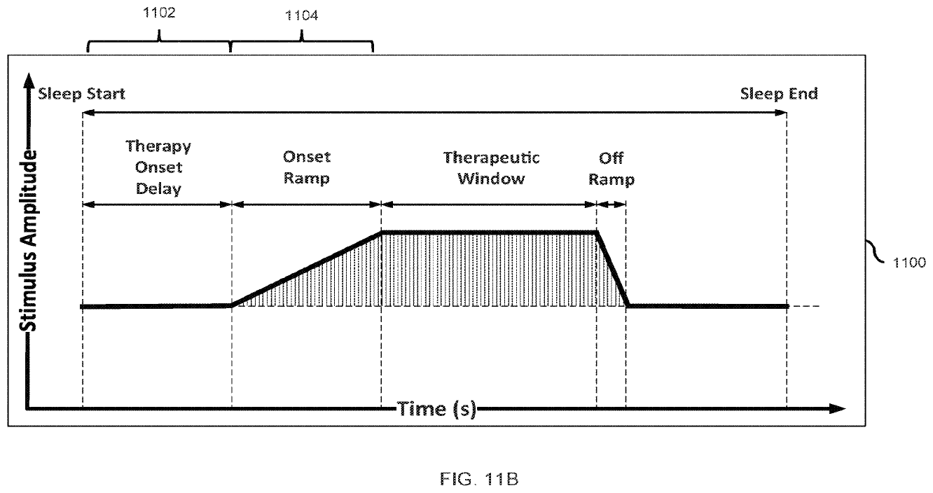

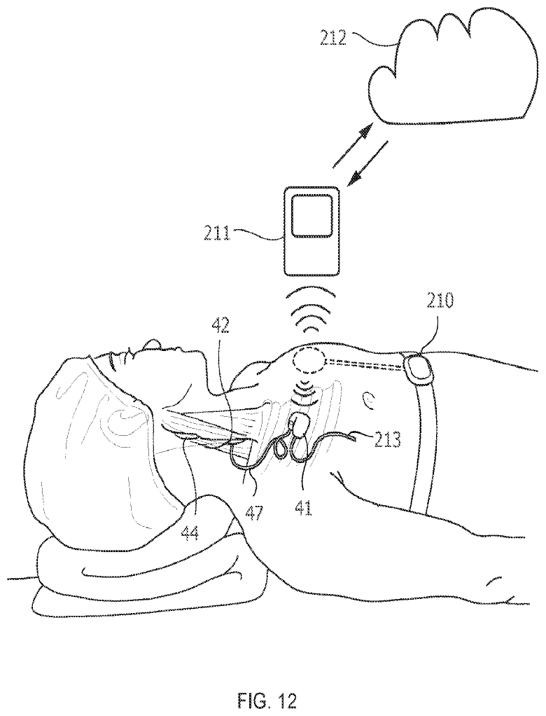

### Description of FIG. 12: Interconnected Distributed System for Monitor Patient at Rest and During Sleep

FIG. 12 shows a patient in bed during sleep therapy. Patient may be sleeping or resting. The IPG 41 is implanted in the patient chest and connected to the electrode system 42 by the stimulation lead 47. Electrode system 42 is in electric contact with the phrenic nerve 44. The electrode system 42 may be an electrode cuff or a paddle electrode. In some examples, the electrode system may include sensors such as an EMG, inertial, microphone, and/or other transducer sensors. In certain examples, the sensors may be integrated with the electrode system (e.g., in the neck). The patient is equipped with a wearable monitoring system 210 in wireless communication with the IPG 41. The wearable monitoring system 210 can include, or be configured to implement, a medical implant communication system (MICS) in order to carry out wireless communication with IPG 41 (e.g., up to 2 meters away from the IPG). The wearable monitoring system 210 may also in communication with a bedside monitor device 211 that can also be a MICS communication device. The monitor device 211 may include a transceiver that allows for data communication, such is an internet communication, with the cloud computing system 212. Data that is communicated to computing system 212 may be stored in non-transitory storage to allow for sharing of data with a physician. The physician may provide data to modify a therapy plan that can be communicated back to the IPG 41, bedside monitor, and/or wearable monitoring system 210 for storage in non-transitory memory of any or all thereof. An updated therapy plan may then be executed by the electronics of the IPG (e.g., in the real time). In some examples, the bedside monitor 211 device may be placed in the pocket of the patient's clothing or placed under the matrass or under the pillow.

The implanted part of the system may include a sensing lead 213 connected to the IPG and tunneled under the skin of the patient's chest to enable improved sensing of muscle EMG, thoracic impedance or acceleration from breathing.

Different communication techniques may be used to facilitate communication between the IPG 41 and other devices that are in communication with IPG. In some examples, an inductive link may be used. An inductive link has a long history of providing reliable communication with pacemakers, ICDs, IPGs, and the like. However, inductive communication can suffer from range (e.g., the maximum separation between the two coils, one inside the body and the other outside the body, must not exceed 6 cm) and data rate limitations (e.g., approximately 100 kbps). Such limitations can be problem when, for example, a patient is sleeping as the link may require realignment to account of movement of the patient while they are asleep. Accordingly, while inductive links may remain relevant for certain types of devices and use cases, other (e.g., future) may user other communication techniques that allow for faster communication over longer distances.

Other communication techniques that may be used in certain examples to allow for communication to/from the IPG 41 include the Medical Implant Communications Service (MICS) that operates in the 402-405 MHz band. MICS allows for higher-speed, lower power, non-voice transmissions to and from implanted medical devices such as cardiac pacemakers and defibrillators. This band has good conductivity in the human body, a higher data rate, and a communication range up to 2 m.

Another communication technique that may be used in certain examples to allow for communication to/from the IPG 41 includes is the Medical Device Radiocommunications Service (MedRadio) that operates in the 401-406 MHz range. The creation of the MedRadio Service incorporated the existing MICS spectrum at 402-405 MHz along with added additional spectrum at 401-402 MHz and 405-406 MHz for a total of five megahertz of spectrum for implanted devices as well as devices worn on the actual body.

It will be appreciated as other communication techniques are developed that such techniques may be employed in connection with the examples described herein.

Another aspect to the system described in connection with FIG. 12 is the measurement of respiration/respiratory activity of the patient. Respiratory activity causes a visible and measurable motion to the chest wall. In certain example embodiments, radar technology can be used to conduct non-contact and non-invasive measurements of respiration. When using such techniques, a radar device is aimed at the chest of a patient, and the resulting motion is recorded and processed to obtain a rate of respiration. In some instances, the use of radar technology may eliminate the need for both implanted and wearable sensing of respiration. In some examples, a bedside radar can be integrated into or connected with the bedside monitor 211 and transmit respiratory cycle information to the IPG using, for example, a MICS communication link in real time. The IPG may then apply stimulation energy based on the timing synchronization signals from the bedside monitor 211.

It is appreciated that non-contacting technology for body motion monitoring is rapidly evolving and becoming more advanced and available. Examples include U.S. Pat. No. 8,454,528. In certain examples, detection of paradoxical movement of the chest wall (where the lung volume may get reduced during the inspiration) can be used as a surrogate variable for inspiratory airway resistance and negative air pressure in the airway distal to the obstruction. This variable may then be used in connection with automatic adjustments of stimulation energy being delivered to a patient via an IPG. Heart rate can also be detected by non-contacting sensors. For example, impulse-radio ultra-wideband (IR-UWB) radar may be used to recognize cardiac motions in a non-contact fashion. Such sensors can be used to measure the heart rate (HR) and/or rhythms using an IR-UWB radar sensor and thus be used to detect/determine rest, falling asleep, periodic hyperpnea, and/or arousal events of a patient. Such techniques may be simpler and/or more advantageous (e.g., than electrocardiogram connection with certain example embodiments.

In any event, various sensors and other devices may be used to obtain respiratory data from the patient. Such data may be obtained by the external wearable 211 or the bedside monitoring device 210. The data can be used to generate commands or other data than are then used to cause the IPG to act or change its operation. For example, commands that are generated based on the processed respiratory data may be communicated to the IPG that cause the IPG to increase the stimulation energy that is being applied it needs to be reduced to commands and transmitted to the IPG to act upon the collected data, since ultimately it is the IPG that controls stimulation flow of stimulation energy to the phrenic nerve.

The maximum allowed energy for each sleeping position for the patient may not be a constant night to night. It may be increased or decreased by the physician remotely or by the logic in the device automatically. On the first night or over several nights after activation maximum energy level can be set to some fraction of the maximum tolerable limit determined during the patient's office visit or test night in the sleep lab (e.g., in connection with FIG. 10B).

Maximum ramped energy can also be set to some preset fraction of the device operating range that can be, for example, 1.0 to 5.0 mV. For example, the maximum energy level for the first night can be set to 50% of operating range. The next night (that can be second night) energy level can be increased to 51%, next night after to 52% followed by further increases of 0.05-5% every night depending on the treatment plan. For example, on initial nights increments may be set to a larger value and then gradually decreased following an asymptotic trajectory pseudo-infinitely approaching the maximum value at the diminishing rate. In this way the patient's central nervous system may be expected to gradually adapt to higher energy levels and new, higher tolerability thresholds can be established gradually and without frequent visits to the clinic.

Automatic telemetry can store relevant parameters such as delivered energy and corresponding physiologic respiratory parameters: motion, posture, breathing, and oxygenation patterns. These parameters may be communicated to the physician or a central analytical facility to supervise therapy. In some examples, a physician can intervene by suspending, reversing, or adjusting, for example slowing down or speeding up the adaptation ramp. There are clear advantages in removing some sensing and control functions from the IPG and redistributing them between outside components that communicate wirelessly with each other and the IPG. The IPG can be made simpler, more reliable, smaller, and with betterer battery life. Additionally, the surgical procedure can be simplified. At the same time the quality of the data signal regarding the patient's body may be improved since the IPG may have limited access to the patient's respiratory system.

Different embodiments of the novel system for optimization of phrenic nerve stimulation parameters to treat OSA may be a distributed system comprising of an IPG in electric communication with the cervical phrenic nerve and components external to the body. The system is capable of delivering controllable excitatory nerve stimulation pulse trains at precise time intervals where stimulation causes diaphragmic contraction of variable strength ranging between twitch capture to fused muscle. In some examples, the system further includes a bedside monitoring controller that is in wireless communication (e.g., continuously) with the IPG. The bedside controller can remote to the patient at the distance of more than 6 cm and up to 2 meters (e.g., with in the MICS range of communication). In some examples, the bedside controller (or another device in communication with the bedside controller) incorporates a mm wave range radar motion detection device that is configured to detect chest motion, estimate breathing rate, separate movement of chest and abdomen, tidal volume and inspiratory time, motion by a patient, position and blood pulsations, and heart rate. The data that is acquired of is then processed by a computer and used to control the start, stop, ramp, increase or decrease of stimulation energy, and adjust stimulation rate—e.g., by communicating with an IPG carry out such changes.

Paradoxical movement of the chest wall (where the lung volume may get reduced during the inspiration) can be used as a surrogate variable for inspiratory airway resistance and negative air pressure in the airway distal to the obstruction. Paradoxical movement can be explained by the reparatory pump inhaling against the elevated upper airway resistance or closed airway. Large magnitude of negative pressure can be achieved in the chest under this circumstance, especially if patient is hyperventilating in response to increasing blood CO2 and hypoxia. Chest muscles and structure cannot resist this negative pressure and chest paradoxically collapsed while the abdomen bulges out. These patterns can be detected by respiratory belts, accelerometers, or non-contacting radar-based motion detectors. A paradoxical movement index can be derived. This calculated variable is used to make automatic adjustments of energy delivery level or timing as described herein.

### Description of FIG. 13: Implantable Pulse Generator (IPG) System Design

FIG. 13 schematically illustrates IPG 41 and is suitable for implanting into a patient with OSA to stimulate phrenic nerve according to the techniques described herein. A phrenic nerve stimulator can be embodied within IPG 41. In certain instances, IPG 41 is an example of a phrenic nerve stimulator. The IPG is similar in hardware design and construction aspects to commercially available implantable pulse generators/implantable neurostimulators, which may be obtained from suitable manufacturers such as Integer® Holdings Corporation.

IPG 41 includes a header 301 for connection to at least one stimulation lead 47 and an optional sensing lead 213. The header can include one or more connection ports (described below). The IPG 41 includes a hermetically sealed housing 202 for containing an electronic circuitry 303 (e.g., the electronics) and a suitable hermetically sealed battery 304. In some examples, the battery 304 may be rechargeable using wireless energy transfer. In some examples, the IPG may include other sensors as part of the IPG 41. Such sensors may include an accelerometer, an oxygen sensor, a vibration sensors, a sound sensor (e.g., a microphone), or the like. Alternatively, or additionally, sensors may be incorporated in a distal portion of the sensing lead 213 or a distal portion of the stimulation lead 47 with the electrical connection to the electronic circuitry 303 internal to the IPG 41. The standard implantable connectors may be similar in design and construction to the low-profile IS-1 connector system (per ISO 5841-3) used in cardiac pacemakers. The IS-1 connectors have been in use since the late 1980s and have been shown to be reliable and provide easy release and re-connection over several implantable pulse generator replacements during the service life of a single pacing lead.

According to one desirable technical feature, the IPG desirably uses (e.g., as, or part of, 303) a standard, commercially available micro-power, flash (in-circuit programmable) programmable microcontroller or processor core in an application specific integrated circuit (ASIC). This device (or possibly more than one such device for a computationally complex application with sensor input processing) and other large semiconductor components may have custom packaging to reduce circuit board real estate needs.

The IPG is controlled using microprocessors with the embedded resident operating system software (code). This operating system software may be further broken down into subgroups including system software and application software. The system software controls the operation of the IPG while the application software interacts with the system software to instruct the system software on what actions to take to deliver an appropriate amount of energy to the phrenic nerve at the appropriate time. The inventors realize that multiple platforms with different system software can be compatible with the application software techniques discussed herein.

The electronic circuitry 303 includes a wireless transceiver. This enables wireless telemetry communication with external systems and therapy controllers that can be wearable, handheld, or bedside devices (e.g., those that are commonly not implanted in the patient).

As an illustrative example, IPG 41 may be responsible for detection of respiration and calculation of respiratory rate via the sensing system, determination of the start time and duration of a stimulation signal, and delivery of a controlled electrical stimulation signal sequences (pulse trains) via the stimulation lead 47. A wearable monitoring system 210 can then be in wireless communication with the IPG and the bedside monitor 211 can be in data communication with loud computer system 212 to enable sharing of data with the physician and modification of therapy plan that can be stored in the memory of IPG and the bedside monitor and executed by the electronics of the IPG in the real time. The IPG may also record and transmit therapy history data (device settings, status, measured data, device use, respiration data, stimulation delivery data, statistics based on motion and sleep time, measured signals, etc.) and implement the patient therapy plan.

As illustrated by FIG. 13, the header 301 forms the top portion of the IPG and may be molded of a polymer that is hermetically sealed to the housing 302. In some examples, the housing 302 may be a formed titanium casing. As mentioned in the context of respiration sensing, the housing may be used as an electrode for bioimpedance signals including respiration measurement. Similarly, electrode systems on leads may be used as an electrode for bioimpedance respiration measurement. For example, the housing may comprise current emitting and voltage sensing electrodes for respiration detection. Alternatively, separate electrodes may be included in the header of the device from which to sense or stimulate.

As noted above, the header 301 may include one or more ports. In the illustration example of FIG. 13, there are two ports: one sensing lead port 305 (labeled “sense”) for receiving the proximal connector of the sensing lead 213 and one stimulation lead port 306 (labeled “stim”) for receiving the proximal connector of the stimulation lead 47. More ports can be added for other leads.

The port configured to receive a stimulation lead 47 may include two set screws (labeled “−” for cathode and “+” for anode) with associated set screw blocks and seals for mechanical and electrical connection to corresponding contacts on a commonly used proximal male plug-in connector of the lead. This design and configuration of the lead-header interface is accepted as standard for devices of this type.

Similarly, the port that is configured to receive a sensing lead includes set screws for current emitting electrodes and voltage sensing electrodes with associated set screw blocks and seals for mechanical and electrical connection to corresponding contacts of the proximal connector of the sensing lead 213. Seals are located between electrical contacts as well as between the distal-most electrical contact and the remainder of the proximal connector assembly. These seals electrically isolate each contact. The header may also include suture holes for securing the IPG to subcutaneous tissue such as muscle fascia using sutures when implanted in a subcutaneous pocket.

During operation, the IPG 41 generates the stimulation output for delivery to the phrenic nerve by way of the stimulation lead 47 in accordance with one or more stimulation parameters. For this purpose, the IPG has a bipolar stimulation output channel corresponding to the stimulation port, with the channel providing a pulse train of biphasic constant current pulses with a frequency range of 20 to 50 Hz, typically at or about 30 Hz, a pulse width range of 30 to 215 μs, typically at or about 150 μs an amplitude range of 0.4 to 5.0 mA, typically 1.0 to 4.0 mA and a stimulation duty cycle range of 30-70%, typically 40-50% by way of example, not limitation. These ranges may depend on the individual patient and on the configuration of the electrode system such as a nerve cuff or a paddle electrode. In certain examples, the burst frequency range or the duration of individual pulses may be determined or otherwise calculated to be as low as possible to generate a smooth contraction, while also selected to conserve battery power. In certain examples, an optimum calculation of the values for the parameters may be performed. The resulting values may then be set within a margin (e.g., 1%, 2%, 5%, 10%, etc.) of those calculated values. Accordingly, in certain examples, the values may be calculated to be, for example, “no longer than needed.”

As shown on FIGS. 13 and 14 and in the accompanying description of the IPG, component design, parameters values and component configurations are given by way of illustration, as a possible example in certain example embodiments, not limitations. Implantable nerve stimulators can evolve rapidly, and traditional technologies are expected to give way to battery-less and leadless stimulators. These improvements are designed to reduce size and increase longevity and reliability of stimulators rather than alter their function in a substantive way.

Impedance sensing is optional for some embodiments of the invention. The IPG circuitry may generate the excitation signal and measures voltage by way of the respiration sensing lead 213 for bioimpedance respiration detection. For this purpose, the IPG has a respiration sensing channel for acquisition of bioimpedance sensing on the desired vector. The vector can be between the sensing lead electrode, the stimulation lead 47 electrode, and the casing (case) of the IPG implanted in the chest.

In certain embodiments, the IPG 41 measures bioimpedance via the port 305, with the electric connections inside providing a small excitation current (“I”) and measuring voltage (“V”). The excitation signal may comprise a 10 to 50 KHz biphasic constant current pulse, with the positive and negative phases of each biphasic pulse having amplitude of 500 μA. Current (“I”) may be fixed by the circuit, allowing voltage (“V”) to be a relative measure of impedance (“Z”), which corresponds to movement of the muscles, lung, airway, and other structures to produce a signal indicative of respiratory activity.

### Description of FIG. 14: Electronic Circuitry

FIG. 14 schematically illustrates the electronic circuitry 303 that may be contained within the IP41. The electronic circuitry 303 may be, or include, a circuit board with a microprocessor (also called a hardware processor herein), memory, I/O, analog to digital (A/D) converter and the like. Any or all of electronic circuitry 303 may reside in the sealed casing 302 of the IPG 41.

The microprocessor 400 is used to control telemetry communications with the external parts of the IPG, operate sensing circuits to monitor motion and respiration, control delivery of output stimuli, monitor an accelerometer (411), magnetically sensitive proximity sensor (e.g. a reed switch) (408), and the real-time clock (409). The microprocessor includes or is coupled to (e.g., as part of the same integrated circuit or on the same silicon chip) RAM (e.g., volatile memory), flash memory (e.g., non-volatile memory), analog to digital (A/D) converter, timers, serial ports, digital I/O, and the like. Microprocessor 400 (e.g., a hardware processor) may be, or form part of, a controller/microcontroller as used herein.

In certain examples, the microprocessor 400 may be composed of several dedicated microprocessors communicating via a serial link. Different functions (e.g., stimulation, monitoring and telemetry communications) may be divided among the various different microprocessors) In some examples, one microprocessor may be used to carry out such functions.

The telemetry interface circuits may consist of a tuned telemetry coil circuit 407 and a telemetry driver/receiver circuit 410 to allow digitally encoded communication between the external components and the microprocessor. As an alternative to telemetry coils and an inductive link, RF antennae with associated circuitry may be used to establish a RF link to provide for longer distance telemetry. The proximity sensor or switch 408 provides a means for the IPG 41 to be controlled by using a magnet of a Near Field Communication device (NFC) placed in close proximity. The real-time clock 409 provides the basic time base (e.g., 768 Hz) for the IPG electronic circuitry 303 as well as a clock (year, day, hour, minute, second) which can be used to control the scheduled delivery of therapy. The clock 409 is also used to time stamp information about the operation of the system that can be recorded on a sleep epoch, hourly, nightly, weekly, or monthly basis.

The bioimpedance respiratory sensing circuit is comprised of two main parts: the excitation current source (output) and the voltage sensing circuit (input) 415. Respiration can be detected using a 3 or 4-wire impedance measurement circuit. In a 4-wire measurement, an excitation current is driven through a pair of electrodes, and the resulting voltage is measured on a separate pair of electrodes. In one embodiment of a 3-wire measurement, the IPG housing (case 302) may be used as both an excitation and sensing electrode. The excitation current circuit delivers bursts of biphasic pulses of low level (e.g., 450 uA) current to the selected electrode pair every 100 ms during sensing. The voltage sensing amplifier circuit 415 synchronously monitors the voltage produced by the excitation current on the selected electrode pair. The resulting output signal is proportional to the respiratory impedance (0.07Ω to 10Ω) and is applied to the A/D circuit in the microprocessor 400 for digitization and analysis. Other sensing circuits can include an ECG signal amplifier (not shown) or a pulse oximetry interface.

The stimulation output circuits deliver bursts of biphasic stimulation pulses to the stimulation lead 47. These bursts may be synchronized to the sensed respiratory waveform to deliver stimulation and thus generate negative airway pressure and reflex causing airway opening at the appropriate time. The stimulation output circuits can include an electrode polarity switching network (425/426), a current source circuit 421, and an output power supply 420. The electrode switching network allows for a charge balancing cycle following each stimulation pulse during which the outputs are connected together with no applied output pulse. The timing and polarity of the pulse delivery is provided by control outputs of the microprocessor 400. The microprocessor selects the amplitude (e.g., 0.4 mA to 5 mA) of the output current from the current source circuit which is applied through the switching/pulse shaping network. The output power supply 420 converts battery voltage (from 417) to a higher voltage (e.g., 5V to 15V) which is sufficient to provide the selected current into the load impedance of the lead electrode system, which can be a bipolar system or a monopolar system with the IPG casing 302 used as current return electrode. The microprocessor 400 may measure the voltage output from the electrode resulting from the delivered current and the load impedance. The microprocessor 400 divides the output voltage by the output current resulting in a measure of the load impedance (e.g., 400Ω to 2800Ω) which can be an indicator of integrity of the lead electrode system and the conditions of the surrounding tissues.

The system may comprise an implanted rechargeable battery (not shown) and an external controller including charging circuitry, a rechargeable battery coupled to the circuitry, and the circuitry adapted for wireless telemetry and energy transfer, and a charging coil coupled to the external controller for generating the radio frequency magnetic field to transcutaneously recharge the rechargeable battery. During a rechargeable battery recharge period, the external controller may be adapted to be carried by a user with no connection to a power main to allow the user to be completely mobile. Rechargeable IPG batteries and circuits are well understood and available from OEMs; they have certain advantages and disadvantages and are often an issue of preference, rather than necessity.

In some examples, the IPG 41 (or lead connected to the IPG) may contain an oxygen sensor to monitor oxygen levels, for example during a night therapy session. The generated signal may be used to monitor efficacy of the therapy. Alternatively, or additionally, the generated signal may be used to cause a change in stimulation delivery settings during a therapy session. For example, the IPG may be programmed to increase stimulation when oxygen de-saturations are detected at a programmable threshold rate and/or severity. In addition, the IPG may turn stimulation on once de-saturations are detected, wherein thresholds of rate and severity are programmable. Desaturations may act to indicate the sleep state or wakefulness. In a similar manner, electroneurogram (ENG) may be used to monitor nerve activity, which may also be indicative of sleep state and/or wakefulness. EMG can be used to monitor muscle activity that may be indicative of spontaneous respiratory effort. The IPG may use the indication of sleep state or wakefulness to change stimulation settings. For example, stimulation may be increased when the patient is estimated to likely be in the N3 or REM sleep. Stimulation level may be decreased or turned off during stage N1 or wakefulness.

As illustrated by FIG. 12, all sensors need not be implanted in the patent's IPG and implanted sensors electrically connected to the IPG but can be distributed between the internal and external (to the patient) and be provided by different components of the overall nerve stimulation system.

The IPG circuitry may contain inertial sensors such as a three-axis accelerometer 411 that can be used to determine the patient's body position (supine, prone, upright, left, or right side) and/or detect motion events (wakefulness). The accelerometer may include gyroscope hardware and firmware. It can measure rotation rate, and acceleration of IPG with high accuracy. These data may be used to change stimulation settings or inhibit output. For example, the IPG may be programmed to increase stimulation intensity when the patient is in specific body positions (e.g., supine, a more challenging position). The IPG may segregate recorded therapy statistics (e.g., cycling detector events, oxygen desaturations) with respect to body position. For example, a patient's cycling detector may record very few events in the lateral position and many events in the supine position, indicative of the patient being treated in the lateral position.

The bioimpedance respiration signal (“Z”) is generated by dividing the change in measured voltage (“V”) by the excitation current (“I”). It may index diaphragm movement, expansion and contraction of a lung and airway over time and therefore is an accepted good measure of respiratory activity. It may be used to estimate in real time, with known imperfections, respiratory effort, respiratory rate, respiratory (tidal) volume, minute volume, etc. If the excitation current (I) is constant or assumed constant, then the bioimpedance (Z) is proportional to the measured voltage (V), and thus the voltage (V) may be used as a surrogate for bioimpedance (Z), thereby eliminating the division step. As used in this context, diaphragm movement includes movements and shape changes of the diaphragm, lung, large airways, and adjacent tissue that occur during normal breathing and during obstructed breathing. The bioimpedance waveform may be filtered to reduce noise and eliminate cardiac artifact, clarifying positive and negative, expiration and inspiration peak occurrence. The signal may be filtered using a first order low pass filter. Alternatively, a higher order filtering approach could be utilized to filter the signal. The (positive or negative) peak of the impedance signal corresponds to the end of the inspiratory phase and the beginning of the expiratory phase. If the signal is normal, the positive peak is used; and if the signal is inverted, the negative peak is used. The beginning of the inspiratory phase occurs somewhere between the peaks and may not be readily discernable. The impedance signal typically provides a reliable timing event for end-inspiration and begin-expiration event. The rest of the breathing cycle may need to be extrapolated based on the patient's history.

Body motion is often indicative of patient wakefulness, can be detected by the accelerometer and may also change the bioimpedance signal (Z). Different thresholds of sensitivity may be utilized such that minor movements are not confused with major motion events such as rolling from side to back in bed, sitting up, standing up, or walking around. When a motion event is determined, stimulation may be turned off or turned down until motion stops or for a programmable duration of time. The frequency and duration of these motion events may be recorded in device history. The accelerometer could be utilized in a similar fashion alone or in combination with impedance to detect and record motion events.

Waxing and waning of the bioimpedance signal (Z) is often indicative of apneas or hypopneas. Generally referred to as cycling, this pattern may be detected, for example, by assessing trends of increasing and decreasing average P-P amplitude values. Different thresholds of sensitivity may be utilized such that minor changes in P-P values are not declared cycling events. When cycling is detected, stimulation parameters may be initiated or changed (e.g., increased intensity, increased duty cycle, etc.) to improve therapy. The frequency and duration of these cyclic breathing patterns may be recorded in therapy history. These values may be used as an indicator of how well the patient is being treated, providing an estimate of AHI.

The IPG may be programmed to change stimulation level between therapy sessions, days, or other programmable value. The stimulation level may be recorded alongside therapy session data, for example cycling rate, oxygen desaturation frequency and severity, stimulation time, variations in respiratory rate, variations in respiratory prediction, etc. Processor 303 is equipped with different computer memory types that can store program code, settings, and patient data in areas with different electronic ways and speeds of updating the contents of memory.

Synchronization to Breathing Vs. Entrainment of Breathing

Triggering the negative pressure reflex based physiologic mechanism may be achieved by diaphragmatic stimulation applied, for example, every breath or every second breath during the late exhalation-early inspiration phase. It can also be applied at any other time during inspiration with likely lesser effectiveness. If applied during exhalation it is not likely to be as effective and may be detrimental by extending expiratory time through the mechanism of the Herring-Bruer inflation reflex.

In people without OSA, inspiration is typically 25-50% of the respiratory cycle, with variations in respiration rate being common. Variations may cause actual inspiration timing to differ from breath to breath. The hypoglossal nerve usually or naturally activates approximately 300 ms before inspiration and remains active for the entire inspiratory phase indicating true beginning of the respiratory cycle. To mimic this natural physiology, it is desirable to deliver stimulation to the phrenic nerve during the late expiratory phase and early inspiratory phase, with the brief pre-inspiratory period of about 300 ms. Since variability is expected, to maximize stimulation coverage of actual inspiration, it may be advantageous to account for this variability by centering stimulation on the predicted inspiration.

Technical ways to synchronize stimulation to breathing have been proposed including sensing based on impedance, accelerometry, breathing sounds and thoracic and airway pressure signals. As the beginning of inspiration can be harder to detect, prediction and/or extrapolations based on peak inspiration timing and known patient history can be used in connection certain examples.

Thoracic impedance requires tunneling of sensing leads and is dependent on the current path vector. This complicates both device design and surgery. Both thoracic impedance and accelerometers can detect chest motion, but not generally the airflow and therefore have problems with the detection of inspiration when the airway is closed or restricted by OSA. This is particularly true in the mixed and obstructive apnea with both normal and paradoxical chest motion present during breathing. Systems tend to overreact during the hyperpnea phase of the OSA cycle, when breathing rate can accelerate dramatically. Devices respond to motion, coughing, sneezing and other signals that are not real inspiration but can trigger detection circuits and make a patient uncomfortable.

Alternative to synchronization is operating the device (e.g., IPG 41) in an “asynchronous mode”. This mode relies on the patient's tendency of synchronizing or at least “phase locking” to external stimulus during sleep. For example, physiologic oscillators tend to phase lock to external stimuli. This doesn't always imply exact synchronization. A good example is synchronization of sleep to daylight cycle. An average person falls asleep sometime in the evening and wakes up sometime in the morning. Similar patterns are seen in patients that “entrain” to mechanical ventilation.

“Phase locking” occurs when the inspiratory efforts of the patient occur at a specific phase, or specific phases, of the ventilator cycle, and the inspirations are periodic in time. This situation can also be called respiratory “entrainment” or synchronization. A phase-locking pattern can be associated with a ratio of ventilator frequency to breathing frequency. For example, in 1:2 phase locking there is one stimulation cycle for two respiratory periods.

The mechanism of entrainment is commonly attributed to vagal afferents from the stretch receptors in the lung and airway, but there is a possible contribution from phrenic and intercostal afferents. Regardless of the exact mechanism, entrainment can be proportional to lung inflation, rate dependent, and apply similarly to mechanical ventilation and phrenic stimulation. There is also similarity between entrainment in natural sleep and in spontaneously breathing sedated patients. Phase locking physiology is described by Graves et. al., in “Respiratory phase-locking during mechanical ventilation in anesthetized human subjects”. Am. J. Physiol 1986.

Human subjects entrained 1:1 to mechanical ventilation over a range of ventilator frequencies that are within ±3-5 breaths/min of the spontaneous respiratory rate of each subject. Outside the range of 1:1 entrainment, more complex entrainment patterns were seen. In the case of phrenic stimulation response of central stimulators is similar. Inspiratory activity can be expected to typically precede the stimulation where the stimulation burst rate is greater than spontaneous frequency, and inspiratory activity occurred during or after the stimulation burst part of the breath cycle when stimulation rate is less than spontaneous frequency in sleeping subjects.

The pattern of entrainment including phase relationship depends on the ratio between “natural” respiratory cycle and mechanical rhythm and the ratio of natural tidal volume and mechanical tidal volume.

In the field of mechanical positive pressure ventilation, the phase relationship of inspiration with respect to the inflation by the ventilator is measured as a delay. The delay is the time from the beginning of a mechanical inflation to the onset of a spontaneous inspiration (typically diaphragmatic EMG). The phase angle θ is derived by dividing the delay by the period of the ventilator and multiplying by 360°. Onset of machine inflation is θ=0° when machine inflation and EMG onset occur at the same time. When EMG activity preceded machine inflation, θ is between −180 and 0°, and, when EMG activity occurred during or after machine inflation, θ is between 0 and +180°. In certain examples, similar techniques can be implemented by the phrenic nerve stimulation system for the stimulation of the phrenic nerve.

In published literature studies, in both anesthetized and sleeping subjects, inspiratory activity preceded the positive pressure mechanical ventilation cycle when machine frequency was less than spontaneous frequency (negative phase angle), and inspiratory activity occurred during or after the ventilator-initiated lung inflation when machine frequency was greater than spontaneous frequency in sleeping subjects.

Entrainment of natural breathing rhythms by stimulation of phrenic nerve is known. It was used to treat Central Sleep Apnea (CSA) as is described in U.S. Pat. Nos. 11,065,443; 11,065,443 and 8,233,987.

In certain example embodiments, a method is provided to entrain respiration for the treatment of OSA by application of a stimulation pulse train to the phrenic nerve causing vigorous contraction of the diaphragm during the late natural exhalation/early inspiration part of the breathing cycle to evoke the negative pressure reflex in the airway when the airway is closed.

In connection with certain example techniques described herein, it can therefore be advantageous to entrain breathing at a frequency rate that causes natural inspiratory activity to occur during or after the phrenic nerve stimulation-initiated lung inflation when stimulation frequency is slightly higher than the spontaneous frequency in a patient. This implies knowledge of the natural breathing rate of the patient. We propose determining that during the rest period when a patient is confirmed to be supine and resting and may be asleep or falling asleep but not yet showing periodic breathing and OSA that alters their natural resting breathing pattern.

It is believed that all patients are easiest to entrain initially at the stimulation rate that is close to their natural breathing frequency (natural frequency stimulation rate). For example, the patient may be initially entrained at their natural frequency stimulation rate and then the rate is gradually increased or decreased to achieve optimal timing of airway stimulation and negative pressure reflex activation.

Further, a processor (e.g., 400), such as in the IPG 41, may be programmed to automatically adjust based on a breathing rate to achieve a desired phase angle θ between 0 and −180°, such as between 0 and −90°, where stimulation is applied after natural inspiration during most breathing cycles. The phase angle is the phase shift between the breathing rate caused by stimulating the phrenic nerve with the IPG 41 and the natural breathing rate with no phrenic nerve stimulation. The automatic adjustments to the breathing rate to achieve a desired phase angle θ between 0 and +180°, such as between 0 and +90° where stimulation is applied before natural inspiration during majority of breathing cycles. Some patients may benefit from the former modality of entrainment and others from the latter one depending on the individual characteristics of the patient.

The EMG activity may be difficult to detect in the home setting, but the delay time from the beginning of a phrenic nerve stimulation inflation to the onset of a spontaneous inspiration can be detected using EMG, transthoracic impedance or accelerometry.

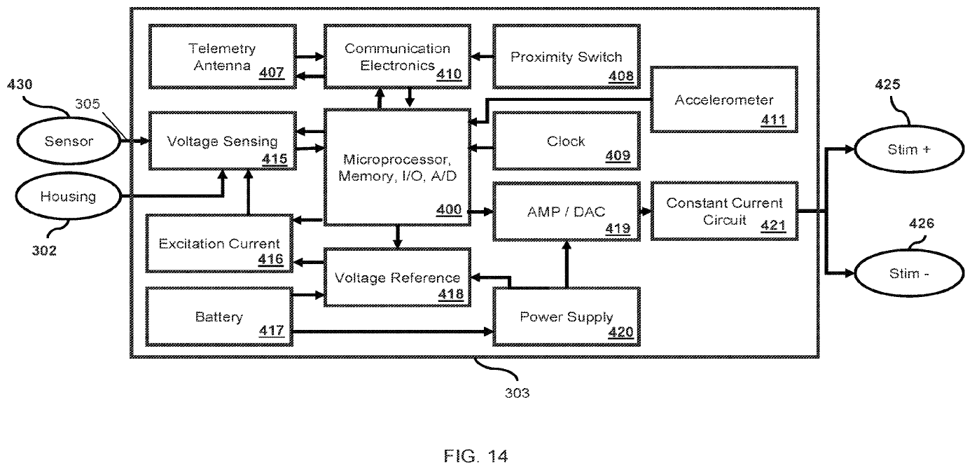

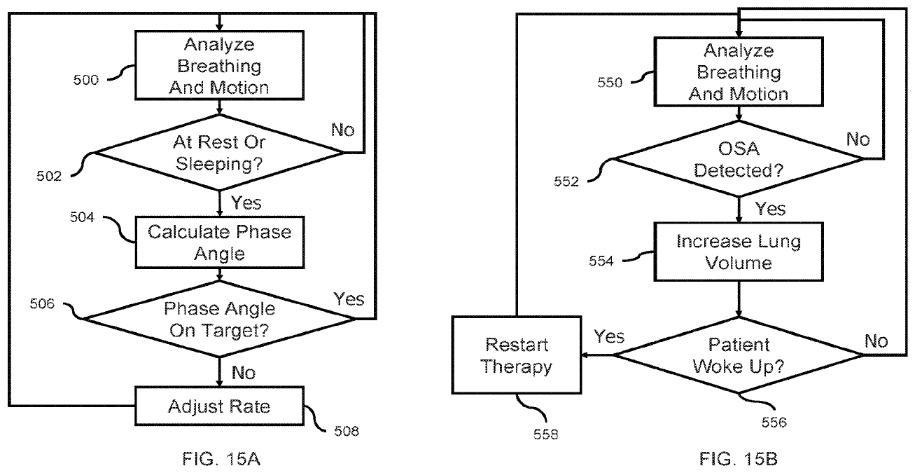

### Description of FIGS. 15A, 16A, and 16B: Optimize Use of Negative Pressure Reflex

FIG. 15A illustrates a process (e.g., an algorithm) that can be implemented in the system to optimize use of negative pressure reflex in the sleeping or resting patient. Entrainment is a phenomenon in which two oscillators interact with each other, typically through physical or chemical means, to synchronize their oscillations. This phenomenon occurs in biology to coordinate processes from the molecular to organ and organism scale and is well described in scientific literature.

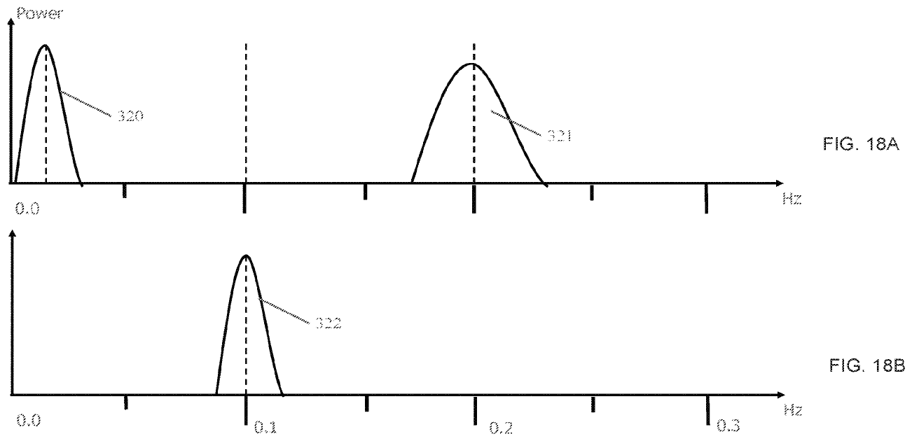

Patient is entrained to the phrenic stimulation and entrainment is confirmed using one of the available methods, such as spectral analysis or Arnold Tongue plot. At 500, the breathing patterns and/or motion of the patient are analyzed. At 502, and based on the analysis at 500, the process determines if the patient is resting or sleeping. If the patient is not resting or sleeping, then the process loops back to 500 to continue monitoring the patient. If the patient is determined to be sleeping or resting, then the processing moves to 504 and the phase angle is calculated using breathing signals. Note that in certain examples, the phase angle may be calculated at 500. In any event, stimulation may be started or otherwise used (e.g., as discussed herein) at a rate that is based on (e.g., as close as possible to) the averaged natural breathing rate. After the entrainment is confirmed, for example by spectral analysis (described below in connection with FIGS. 18A and 18B), stimulation rate can be increased or decreased (e.g., slightly) based on (at 506) a determination that the calculated phase angle is not on target to thereby achieve the desired timing. If the phase angle is on target, then the process returns to 500 and monitoring the patient. If, however, the phase angle is not on target, then the stimulation rate may be adjusted by increasing or decreasing the rate. For example, a stimulation rate can be set 2-3 breaths below a natural breathing rate. The phase angle will be expected to be negative. Accordingly, the stimulation rate can increased in steps at 508 until the phase angle changes polarity and stimulation precedes the natural inspiration by the desired delay that can be 50-500 ms or 25% of the total natural inspiratory time.

In some embodiments, the elements shown in FIG. 15A may each be performed by an IPG (e.g., the electronic circuitry thereof) In other embodiments, some elements may be performed by the IPG and others may be performed by other devices that are in communication with the IPG. For example, the analysis of breathing and motion that is performed at 500 can be by a mobile device or a bedside monitor that then communicates with the IPG to adjust the stimulation rate being delivered to the patient.

FIGS. 16A and 16B illustrate how the stimulation rate can be adjusted to optimize phase locking of breathing using the phase angle between stimulation pulse trains and spontaneous breathing effort of the patient.

FIG. 16A illustrates stimulation pulse trains 220 that are applied at a set rate that may be between 6 and 25 breaths per minute. This rate is below the patient's natural breathing rate. Breaths represented by volume change 221, lag behind the stimulation by a delay period 222. Each breath represents tidal volume that results from the combination of natural effort and effort induced by stimulation.

FIG. 16B illustrates a stimulation rate that is increased in comparison to that shown in FIG. 16A. In FIG. 16B the stimulation pulse train 223 leads to the positive phase shift 225 and acceleration of the breathing rate 224 of the patient. Combined tidal volume is reduced by the patient's central nervous system to maintain minute ventilation and blood gases in the normal range. Stimulation in connection with certain example embodiments occurs mostly during late expiration-early inspiration phase of natural breathing for optimizing the utilization of negative pressure reflex. Zero volume exhaled or inhaled at the beginning of inspiration 226 illustrates the lack of airflow through the closed airway.

FIG. 16B also illustrates a breathing and stimulation pattern that is similar to or represented by patient data from FIG. 7. Specifically, the first stimulation burst 60 is initiated when the patient's airway is closed as evidenced by air flow of zero during period 66. It is understood that, as can be seen from FIG. 7, behaviors of biological oscillators are imperfect and phase angle can change polarity between breaths. The phase lock or entrainment shall be interpreted as a predominant or statistically more frequent behavior when applied to series of spontaneous breaths that occur over hours of sleep during the night. Accordingly, in connection with certain example embodiments discussed herein, when “analyze breathing” or the similar is mentioned, it should be understood that computer controlled logic that is part of or embedded in the system is configured to average, analyze, and otherwise process a series of breaths before changes to stimulation settings such as rate, duration, and length of pulse trains are made. Where rate is mentioned, it is to be understood that this includes the rate of pulse trains or bursts that are composed of individual pulses. For example, pulse trains can be applied at a rate of 10 pulse trains/minute and composed of individual bipolar pulses repeated at 30 Hz frequency (pulses/second). Thus, the terms pulse burst, and pulse train are used interchangeably herein.

### Description of FIG. 15B: Use of Lung Volume

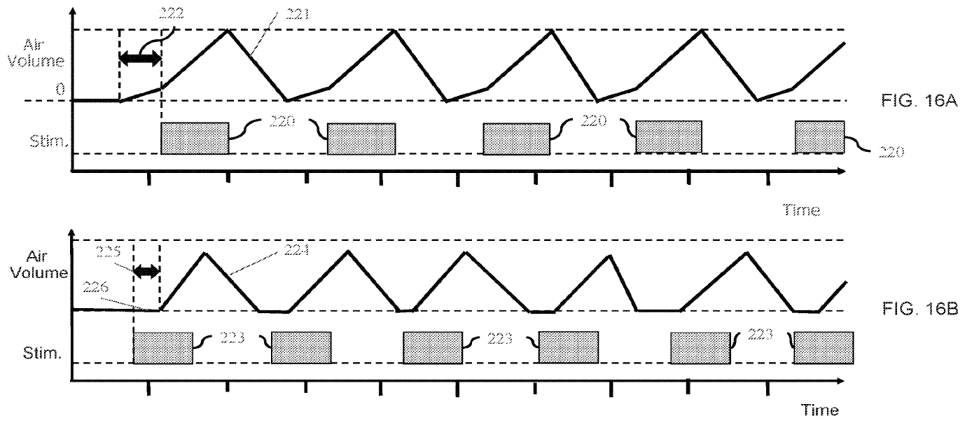

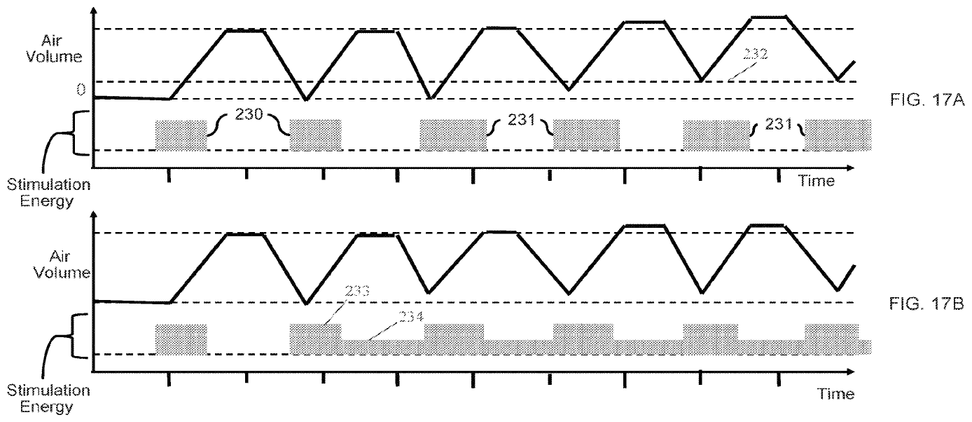

FIGS. 15B, 17A, and 17B illustrate use of lung volume to optimize and improve effectiveness of phrenic nerve stimulation to treat OSA.

The I:E ratio is the ratio of the duration of inspiratory and expiratory phases of a breath. In mechanical ventilation, a “normal” I:E ratio is approximately 1:2. It is important in natural and mechanical respiration to ensure the breath delivery includes adequate time to exhale. Normal inspiratory to expiratory ratios (I:E) of spontaneously breathing patients are usually around 1:3 to 1:5. Meaning, the ratio of time in expiration is 3 to 5 times longer than the ratio of time in inspiration.

As used herein, terms I:E ratio and duty cycle are similar but not interchangeable. For example, I:E of 1:1 corresponds to duty cycle of 50% of the total respiratory cycle. As used herein, the phrenic nerve stimulation duty cycle (duty cycle) means the percentage of stimulation burst duration over the total length of the stimulation cycle. These parameters are part of the IPG settings. For example, if stimulation rate is set to 10/minute and duty cycle is set to 40%, the cycle is 6 seconds long and stimulation burst is 2.4 seconds long.

As used herein, the I:E ratio means a ratio between total inspiration and exhalation time for the patients measured at a mask. It may combine the artificial or stimulation induced inspiration and the natural inspiratory effort. Since in OSA the airway may be occluded, inspiratory time may not correspond to the inspiratory effort one to one.

As used herein, duty cycle can be used to manipulate (e.g., increase) I:E ratio of an entrained spontaneously breathing patient. The purpose of increasing duty cycle is to increase lung volume and specifically increase end expiratory lung volume of the sleeping patient with OSA. This phenomenon is often called “air trapping”.

It is generally recognized that for ventilated patients, substantial deviations from normal I:E ratios are uncomfortable and in mechanical ventilated patients, require sedation. Nevertheless, there are circumstances in which increasing the I:E ratio is well tolerated and therapeutic. Similar or the same aspects may apply to the phrenic nerve stimulation techniques discussed herein. For example, increasing a duty cycle from 30% to 50% may be beneficial, while increasing the duty cycle from 50% to 70% may be uncomfortable to the patient. Accordingly, these settings are individualized and may be included in the profile for a patient.

Lengthening the inspiratory time (e.g., I:E of 1:1) increases mean airway pressure. As mean airway pressure rises, lung volume increases and atelectatic regions of lung inflate, leading to improvements in oxygenation. But this strategy can have its limits as, for example, the lungs may be better utilized for gas exchange.

In respiratory therapies, such as mechanical ventilation and CPAP, the end-expiratory lung volume (EELV) is the natural functional residual capacity (FRC) plus lung volume increased by the applied positive end-expiratory pressure (PEEP). It is believed that moderate increase of EELV benefits patients with OSA by increasing caudal traction on the airway which makes it more resistive to collapse.

Lung volume is known to fall during sleep. Under these circumstances, increasing the inspiratory duty cycle can stabilize lung volume at wakeful levels by: (1) increasing mean airway pressure, and (2) trapping air inside the lung when insufficient time is allotted for patients to exhale completely. While this maneuver can be uncomfortable in conscious individuals, it is usually well-tolerated during sleep if applied in controlled manner.

Obesity is often associated with reductions in end-expiratory lung volume during wakefulness, which further worsen during sleep and sedation. The drop in lung volume during sleep is known to aggravate upper airway obstruction and nocturnal hypoxemia in these individuals. Deleterious effects of low lung volumes can be reversed by increasing the I:E ratio. Resulting elevations in lung volume effectively maintain pharyngeal patency and mitigate hypoxemia during sleep and sedation. These responses can be exploited to treat obstructive and central sleep apnea, respectively.

### Description of FIG. 17A: Increase Duty Cycle

FIG. 17A illustrates increasing duty cycle of stimulation of one phrenic nerve. The stimulation portion 230 of a first duty cycle is shorter than the stimulation portion 231 of a second duty cycle. By selectively applying different duty cycles (e.g., shorter 230 to longer 231 duty cycles), a controlled increase of end-expiratory lung volume 232 (EELV) may be achieved.

In patients with obstructive sleep apnea, increases in lung volume can exert caudal traction on upper airway structures, thereby preventing pharyngeal collapse and mitigating increase of airway resistance. As lung volume rises, oxygenation also may improve due to concomitant reductions in physiologic shunt, oxygen reserve and improved ventilation/perfusion ratios. Negative cardiac effects from increased intrinsic Positive End Expiratory Pressure (PEEP) and increased pulmonary vascular resistance may be well-tolerated or even negated by reduced hypoxic vasoconstriction in reinflated regions of lung as they re-oxygenate.

Controlling and optimizing I:E ratio of ventilation for therapeutic purposes by changing the stimulation duty cycle may use real time feedback control loops, which can be implemented as a computer-controlled process that is, for example, embedded in software of the IPG. For example, a rise in the patient's intrinsic or entrained respiratory rate could also shorten expiratory time, further impeding exhalation and elevating lung volume. Increase of the stimulation duty cycle may also have a similar effect on lung volume. In this regard, the end-expiratory lung volume could be maintained at the desired level for the optimal breathing rate (one that is phase locked) by controlling the duty cycle.

In certain examples, a relatively healthy person may require a comparatively smaller tidal volume during sleep to satisfy their metabolic needs. As a result, inspired air can be exhaled completely, without air trapping, in a relatively short time. Therefore, in a setting of normal sleep breathing so called reversed I:E ratio may be required to trap air in the lung and increase EELV. The anticipated duty cycle (e.g., as applied to such cases) may be in the range of 50 to 70%. At the same time, some patients, especially ones with heart or lung disease, may have rapid breathing and height expiratory resistance. These patients may trap air at less of a duty cycle. Such data settings can accordingly be individualized to the patient and can be stored in the profile of the patient for later use.

### Description of FIG. 17B: Bilevel Stimulation

In another embodiment, we propose to apply entrainment to the treatment of OSA by application of at least two levels of stimulation: inspiratory level and expiratory level. This can be called “bilevel entrainment”.

Bilevel entrainment is directed to augmenting inspiration, opening the collapsed airway using negative pressure response, setting the breathing rhythm, and maintaining augmented lung volume during expiration. It is intended to maintain natural respiration whilst regularizing the breathing to the rate set by the timing of the stimulation while maintaining two corresponding levels of lung distension: inspiratory volume and end-expiratory “bias” volume.

The duty cycle can be set by the physician or adjusted automatically if too much or too little air trapping is detected. In order to be practical, such therapy needs to be adaptive where both inspiratory and expiratory period stimulation levels can be automatically adjusted based on patient's respiration and body position.

Augmented inspiration may compensate for the reflex induced reduction of tidal volume caused by the increased residual lung volume known as Hering-Breuer inflation reflex. It is understood that entrainment can be applied to every second breath or for a period of time followed by natural rhythm and restoration and reassessment of breathing rate and minute ventilation. Supplementing minute ventilation by increasing tidal volume can be an important part of therapy in patients where it is clinically indicated such as in sleep induced hypoventilation, CSA, and/or obesity.

It is understood that the proposed bilevel entrainment stimulation can treat a variety of conditions that often accompany OSA such as obesity induced hypoventilation, central sleep apnea, and mixed sleep apnea (e.g., an apnea type where airway instability is accompanied by the instability of respiratory drive).

It can be expected that the proposed bilevel entraining stimulation can also be instrumental in titration and auto-titration of phrenic stimulation therapy. Processes in natural and induced respiration are by nature periodic and follow rhythms. This includes central and obstructive apneas and hypopneas. While respiratory signals are often noisy and hard to discriminate, periodicity can be identified in the frequency domain using power spectrum analysis tools. Such tools can be, to a certain extent, immune to random mechanical and electric noise—as well as changes to patient's position.

The stimulation is bilevel and consists of the inspiratory part and the expiratory part that is lower level than the inspiratory part but sufficient to bias the lung and maintain expiratory lung volume above natural state. The bias (exhalation period) energy level may be adjusted in response to respiration analysis. Of specific interest is the intended reduction of the power spectrum density in the very low LF band that reflects apnea hypopneas and directly related to the goal and mechanism of therapy.

Bilevel stimulation is illustrated in the graph of FIG. 17B. The X-axis shows time, and the Y-axis shows both air volume along the top, with stimulation energy along the bottom. FIG. 17B illustrates tidal volume, as in breath-by-breath respiratory airflow integrated over time, inspiration phase followed by expiration. The bottom trace of the graph along the illustrates the applied stimulation energy. Stimulation pulse trains are set to a programmed frequency (e.g., based on the clock and the software in the IPG), which can be between 6 and 20 breaths/min (0.1 and 0.33 Hz), which is approximately the physiologic range where patient's natural breathing can be expected and can entrained by stimulation. The combined effort of the patient and induced diaphragmic stimulation create inspiratory effort and generates a corresponding tidal volume that is displayed along the upper part of the graph.

Stimulation during inspiration 233 corresponds to the inspiratory level of the stimulation energy which reflects the IPG generated pulse train characterized by certain frequency, duty cycle, and electric current directed towards the phrenic nerve. Contrariwise, the expiratory stimulation level 234 is lower than inspiratory level and selected to maintain bias of the lung and the certain desired end expiratory lung volume to prevent full deflation of the lung, to keep it inflated and improve airway resistance to occlusion and collapse according to other aspects described herein.

In OSA, the respiration diminishes over time until an apnea manifests. The bias stimulation level can be increased until respiration resumes. In the practical embodiment of an auto-titration algorithm, bias stimulation may be increased in advance of apnea, when hypopnea is detected, since it is easier to maintain airway open than to reopen it after it has collapsed completely. It can be set in advance based on the known patient's behavior during the night as recorded in the patient's profile. It may be applied when patient changes position, for example rolls to supine position.

In certain examples, an airflow signal alone may not be sufficient to distinguish between obstructive and central apnea and hypopnea. Accordingly, additional sensing of respiratory effort can be used. For example, trans-lung impedance can be used as an indication of respiration certain vectors can detect paradoxical movement of the chest wall where the lung volume may get reduced during the inspiration. Where spectral analysis is used, these considerations are largely irrelevant since analysis detects periodicity, not magnitude or direction of respiratory effort.

### Description of FIGS. 18A and 18B: Spectral Power Analysis

FIGS. 18A and 18B illustrate spectral power analysis that may be implemented by/on the IPG (e.g., in software or firmware loaded therein) or an external device in wireless communication with the IPG. An advantage of breathing analysis in frequency domain is that it is more sensitive to the rate of breathing and less sensitive to patterns of breathing, which is valuable in patients with OSA that may exhibit paradoxical breathing airway occlusion. Even a highly imperfect signal, such as integrated and bypass filtered accelerometer reading, is likely to produce an accurate estimate of natural breathing rate over time, while being relatively insensitive to occasional signal noise such as coughs or rolls in bed.

Power spectrum can be obtained by performing Fast Fourier Transform (FFT) on 1-10 minutes of digitally acquired respiratory signal data (in this example: chest motion, impedance changes, or breathing sounds). The spectrum can be a power spectrum, a power density spectrum, or a magnitude spectrum.

Power spectrum allows estimation of which periodic frequencies contribute most to the total variance of the signal in the band of interest. The larger the amplitude, the higher the variance. It is understood that there are many techniques for calculating frequency distribution of periodic signals and such techniques may be employed in connection with the example embodiments contemplated herein.

The “spectrum” may be calculated for the range of natural respiratory frequencies that generally are between 0 and 1.0 Hz. In certain example embodiments, the frequency range can be approximately 0.1 to 0.5 Hz is of interest. Where non-physiologic high frequency oscillations are intentionally applied, the ban can be expanded. The selected range is designated a “respiratory frequency band” in connection with use of the techniques herein. Other frequency ranges could be selected, with the selection of ranges based on FIGS. 18A and 18B being exemplary.

Referring now more specifically to FIG. 18A, this graph shows an example of a respiratory power spectrum in a patient suffering from apnea and breathing naturally. The low frequency (LF) power peak 320 corresponds to periodic breathing, apneas, or hypopneas that may be present when patient is resting and not yet asleep in the case of CSA but generally manifest during sleep in OSA. It will be appreciated, that it is generally not important if patient has central or obstructive apnea in this context, since both processes are by definition periodic, since obstructive apneas are periodically interrupted by bouts of compensatory hyperventilation.

Unlike hypoglossal nerve stimulation, phrenic nerve stimulation may be beneficial to both OSA and CSA when applied asynchronously. For example, as illustrated by FIGS. 18A and 18B, spectral analysis may be instrumental in determining an initial stimulation rate that may be set to be around patient's natural breathing peak frequency and in determining magnitude of periodic breathing to use as a basis for the decision to increase energy delivery or change stimulation rate or duty cycle (titrate therapy up).

The Apnea/hypopnea power frequency band is generally contained around 60/hour and 120/hour (0.017 and 0.033 Hz) and integrated power in this band is attributed to periodic breathing peak 320. The high frequency HF peak 321 corresponds to respiration and can be quite diffuse in patients that breath irregularly, but overall is concentrated between 6 and 20 breaths/min (0.1 and 0.33 Hz).

FIG. 18B shows entrained patient's respiration where periodic breathing is resolved. Patient breathing is phase locked (entrained) to the stimulation applied at the constant asynchronous rate of 0.1 Hz (6 breaths/min). Stimulation power peak 322 corresponds to this setting. If there is no capture of the diaphragm, then the peak will not be distinguishable from background noise. Thus, this technique is instrumental in detecting levels 103 and 109 on the titration curve (see FIG. 9). In certain examples, the software that is described herein may calculate or use a percentage of calculated respiratory power concentrated in the designated frequency band, which is normalized to the total power after removal of noise, instead of absolute values.

It will be appreciated that the frequency numbers provided herein are selected for illustration purposes. In practice, a patient's natural breathing and stimulation rate can be closer—for example within 2-3 breaths per minute from each other. This means that the instant breathing rate calculated from breath durations is statistically distributed around the stimulation rate. If the power distribution is skewed—for example towards a frequency that is higher than the stimulation rate—then the natural breathing rate may be higher than the stimulation rate and the stimulation rate may need to be increased.

It will be appreciated that the illustrations are provided by way of example (e.g., they may be somewhat idealized) to help illustrate the principles of spectral analysis. In certain examples, all three peaks may be present, and power may be distributed between them in different proportions. A goal in certain example embodiments, is to reduce LF power of the spectrum and concentrate as much power as practical (without waking the patient or completely taking over natural respiration) in the narrow frequency band centered around the stimulation frequency.

It is appreciated that the different techniques described herein may apply differently and in different combinations to patients with different characters, diseases, underling physiology, and anatomy. Some patients may benefit from some lung volume increase, and some may not. Some patients may benefit from phase locking their breathing to a rate higher than their natural respiration at rest, and some may not.

While the invention has been described in connection with what is presently considered to be the most practical and preferred embodiment(s), it is to be understood that the invention is not to be limited to the disclosed embodiment, but on the contrary, is intended to cover various modifications and equivalent arrangements included within the spirit and scope of the appended claims.

### Additional Embodiments

Additional embodiments that may be implemented can include the following exemplary methods for treating sleep apnea.

Embodiment 1. A method of treating obstructive sleep apnea (OSA) including periodically artificially stimulating at least one phrenic nerve of a patient where the stimulation is applied to the nerve while the pharyngeal airway of the patient is naturally obstructed.

Embodiment 2. A method of treating OSA by periodically stimulating at least one phrenic nerve of a patient where stimulation is applied while the airway is closed or partially occluded.

Embodiment 3. A method of treating OSA by periodically stimulating at least one phrenic nerve of a patient where stimulation is applied while the airway is characterized by increased obstruction.

Embodiment 4. A method of treating OSA by periodically stimulating at least one phrenic nerve of a patient where stimulation is artificially applied while the airway is closed.

Embodiment 5. The method of any of Embodiments 1 to 4, wherein the stimulation includes a stimulation burst initiated while the airway is closed.

Embodiment 6. The method of any of Embodiments 1 to 5, wherein a substantial proportion of stimulation are simulation bursts initiated when the airway is closed.

Embodiment 7. The method of Embodiments 5 or 6, wherein the stimulation burst(s) is applied first at a first energy level sufficient to generate action potentials in phrenic nerve and later at second energy level sufficient to evoke reflex opening of the collapsed airway by activation of upper airway muscles.

Embodiment 8. The method of any of Embodiments 5 or 6, wherein the stimulation burst(s) is applied first at a first energy level sufficient to generate action potentials in phrenic nerve and further at a second energy level sufficient to evoke reflex opening of the collapsed airway by potentiation of a mechanoreflex.

Embodiment 9. The method of Embodiment 9, wherein the mechanoreflex is a negative pressure reflex.

Embodiment 10. A method comprising:
identifying a patient with OSA,
implanting in the patient a phrenic nerve stimulator or connecting the phrenic, and
adjusting stimulation energy applied by the phrenic nerve simulator to a phrenic nerve in a sleeping patient based on detected airway occlusion in an airway the patient.

Embodiment 11. The method of Embodiment 10, wherein the phrenic nerve stimulator includes a pulse generator and an electrical electrode implanted proximate to the phrenic nerve.

Embodiment 12. The method of Embodiments 10 or 11, wherein the adjusting of the stimulation energy includes adjustments made until the airway occlusion is opened in response to the stimulation bursts.

Embodiment 13. The method of any of Embodiments 10 to 12, wherein the adjusting of the stimulation energy continues until airflow is restored in the sleeping patient.

Embodiment 14. The method of any of Embodiments 10 to 13, where airway occlusion is detected by monitoring airflow through air breathing passage in the patient, respiratory sounds of the patient, respiratory effort by the patient, airway pressure and/or oxygen saturation of the patient.

Embodiment 15. The method of any of Embodiments 10 to 14, wherein the stimulation energy is applied during a breath where the breaths are at a rate of 6 to 20 breaths per minute, 8 to 14 breaths per minute, 6 to 15 breaths per minute, or 10 to 15 breaths per minute.

Embodiment 16. The method of any of Embodiments 10 to 15 wherein the applied energy is applied in a duty cycle of 30 to 50% of a breath period, 35% to 40% of a breath period, 40% to 60% of a breath period, or 25% to 60% of a breath period.

Embodiment 17. The method of any of Embodiments 10 to 16, wherein a rate is set based on patient's natural resting breathing rate.

Embodiment 18. The method of any of Embodiments 10 to 17, wherein at least 20% of applied energy includes stimulation bursts coinciding with a natural late expiration early inspiration period of a breath.

Embodiment 19. The method of any of Embodiments 10 to 18, wherein the applied energy includes stimulation bursts are applied during the time of the natural late expiration early inspiration period of the breath detected or predicted based on previous breaths.

Embodiment 20. The method of any of Embodiments 10 to 19, wherein the stimulation of the phrenic nerve generates negative pressure in the airway.

Embodiment 21. The method of any of Embodiments 10 to 20, wherein the stimulation of the phrenic nerve generates a diaphragmic contraction that generates a negative pressure in the airway.

Embodiment 22. The method of any of Embodiments 10 to 21, wherein the stimulation of the phrenic nerve generates a diaphragmic contraction that generates negative pressure in the airway sufficient to trigger a negative pressure reflex.

Embodiment 23. The method of Embodiment 22, wherein the negative pressure reflex activates an airway muscle in the patient.

Embodiment 24. The method of Embodiment 23, wherein the activation of the airway muscles restores patency of the airway.

Embodiment 25. The method of Embodiments 23 or 24, wherein the activation of the airway dilator muscles minimizes oxygen desaturation and/or hypercapnia.

Embodiment 26. A method of shortening the time of an airway occlusion in a sleeping patient comprising periodically stimulating at least one phrenic nerve of the sleeping patient where the stimulation is applied while the airway is closed.

Embodiment 27. The method of Embodiment 26 wherein the stimulation of the at least one phrenic nerve contributes to a trigger of a negative pressure reflex in the patient.

Embodiment 28. The method of Embodiments 26 or 27, wherein the stimulation of at least one phrenic nerve induces contraction of diaphragm in the patient when the airway is collapsed.

Embodiment 29. The method of Embodiment 28, wherein the contraction of the diaphragm creates negative transmural airway pressure downstream of an occlusion site in the airway.

Embodiment 30. The method of Embodiment 29, wherein the negative transmural airway pressure is sufficient to activate a negative pressure reflex in the patient.

Embodiment 31. The method of any of Embodiments 26 to 30, wherein a negative pressure reflex efferent output to airway muscles exceeds naturally negative pressure reflex efferent output occurring in during sleep of the patient.

Embodiment 32. The method of any of Embodiments 26 to 31, wherein contraction of a diaphragm in the patient is sufficient to create a negative transmural airway pressure downstream of an occlusion site in the airway and is more negative than a naturally occurring negative transmural airway pressure occurring during sleep of the patient.

Embodiment 33. The method of any of Embodiments 26 to 32, further comprising shortening a period of airway occlusion in the sleeping patient to prevent oxygen desaturation of more than 3%.

Embodiment 34. The method of any of Embodiments 26 to 32, further comprising automatically adjusting a stimulation current based on at least one of a detected airflow in a breath of the sleeping patient, respiratory sounds of the sleeping patient, respiratory effort of the sleeping patient, airway pressure in the sleeping patient and oxygen saturation of the sleeping patient.

Embodiment 35. A method comprising:
identifying a patient with OSA, and
adjusting stimulation energy to a phrenic nerve in the patient based on detected airway obstruction in an airway the patient and while the patient is sleeping.

Embodiment 36. The method of Embodiment 35 further comprising confirming that the patient remains asleep after the adjustment of the stimulation energy.

Embodiment 37. The method of Embodiment 36 further comprising selecting a stimulation energy which both causes the occlusion to cease and open the airway, and does not awaken the patient.

While the invention has been described in connection with what is presently considered to be the most practical and preferred embodiment(s), it is to be understood that the invention is not to be limited to the disclosed embodiment, but on the contrary, is intended to cover various modifications and equivalent arrangements included within the spirit and scope of the appended claims.

### LIST OF ELEMENTS IDENTIFIED IN FIGURES

Patient 1
pharyngeal airway (pharynx, airway) 2
negative inspiratory pressure in the airway 3
positive pressure outside the airway 4
pharyngeal dilator muscles (e.g., genioglossus) 5
mandible 6
increase of lung volume 7
Soft palate (vellum) 8
velopharynx or velopharyngeal space 9
CNS respiratory center 10
physiologic sensors 11
genioglossus 14
afferent limb of reflex 12
efferent limb of reflex 13
medulla 16
phasic activation of pharyngeal dilator muscles 20
pharyngeal patency 21
compromised pharyngeal anatomy 22
reflex reduction 24
sleep onset 23
reduced response to negative pressure 25
airway closure 26
hypoxia and hypocapnia 27
increase respiratory effort 28
arousal 29
phrenic nerve stimulation 30
augmented negative pressure 31
activation of afferent limb of NPR 32
restored activity of dilators 33
implantable pulse generator (IPG) 41
electrode system 42
diaphragm 43
phrenic nerve 44
lung 45
stimulation system 46
lead 47
handheld computer programing instrument 48
airflow 50
respiratory effort 51
oxygen desaturations during apnea 52
control period when stimulation is turned off 52 54
periods when turned on 53, 55, 56
stimulation current level 55
increased current level 56
50-a First trace in graph showing airflow sensor signals
51-a Second trace in graph showing respiratory belt sensor signals
52-a Third trace in graph showing pulse oximeter measurements
53-a Arbitrary Waveform Generator
first stimulation burst 60
second stimulation burst 61
beginning of effort 62
Inspiration turns into expiration 63
cessation of effort 65
time delay 66
airway opens, beginning of inspiratory airflow 67
obstructed but not closed airflow 69
delay time 71
Inflection point 73
inspiration is terminated by the respiratory 74
exhales point 75
70-a Airflow rate chart
70-b Respiratory effort over time chart
70-c Respiratory effort signal
70-d Air flow signal
Detect sleep onset and OSA 80
Analyze breathing 81
Stimulate phrenic nerve 82
Normal Breathing Restored 83
Adjust parameters 84
Accept parameters 85
Curve of contraction strength vs. stimulation energy 100
Diaphragm contraction strength 101
Stimulation energy 102
Lowest capture level 103
Breathing normalization level 104
Tolerance level 105
Highest muscle tetanic contraction level 106
Therapeutic range 107
Reflex activation level 108
Analyze breathing and motion 150
Patient at rest 152
Increase energy 154
Capture detected 156
Store data & stop test 158
Analyze breathing and motion 170
OSA detected 172
Increase energy 174
Wake up 176
Decrease energy and store data 178
Graph 200
test ramp 201
first therapeutic ramp 202
night rest capture threshold 205
first therapeutic threshold 206
second therapeutic ramp 204
second therapeutic threshold 207
Wearable monitoring system 210
Bedside monitor device 211
Cloud computer system 212
Sensing lead 213
stimulation pulse train 220
volume change 221
Delay 222
Increased stimulation rate 223
Breathing rate 224
Positive phase shift 225
Stimulation portion 230 of first duty cycle
Stimulation portion 231 of second duty cycle
end-expiratory lung volume 232
Stimulation during inspiration 233
Header 301
Housing 302
Electronic circuitry 303
battery 304
Sensing lead port 305
Stimulation lead port 306
low frequency LF power peak 320
high frequency HF peak 321
Stimulation power peak 322
Microprocessor 400
Tuned telemetry coil circuit 407
Proximity sensor or switch 408
Real-time clock 409
Telemetry driver/receiver circuit 410
Three-axis accelerometer 411
Voltage sensing circuit 415
Excitation Current 416
Battery 417
Voltage reference 418
Amp/Digital-to-analog converter 419
Power Supply 420
Current Source Circuit 421
Polarity Switching Network, Positive 425
Polarity Switching Network, Negative 426
Sensor 430
Analyze breathing and motion 500
At rest of sleeping determination 502
Calculate phase angle 504
Phase angle on target determination 506
Adjust rate 508
Analyze breathing and motion 550
OSA detected 552
Increase Lung volume 554
Wake up determination 556
Restart therapy 558
Latency time parameter 1102
Therapeutic ramp time parameter 1104.

1. A system configured to treat obstructive sleep apnea (OSA), the system comprising:
a phrenic nerve stimulator configured to deliver stimulation energy to a phrenic nerve in a sleeping patient;
at least one sensor configured to output one or more signals indicative of an occlusion status in an airway; and
a controller, which includes at least one hardware processor, configured to perform operations comprising:
receiving the one or more signals, and
controlling the phrenic nerve stimulator to deliver the stimulation energy based on the one or more signals.

2. (canceled)

3. The system of claim 1, wherein controlling the phrenic nerve stimulator further comprises:
in response to receiving from the at least one sensor one or more signals identifying that the airway is partially or totally occluded, commanding the phrenic nerve stimulator to deliver, or increase delivery of, the stimulation energy while the airway is partially or totally occluded.

4. (canceled)

5. The system of claim 1, wherein the operations further comprise:
receiving or determining, for a given patient, a diaphragmic muscle contraction threshold sufficient to cause diaphragmic muscle contraction and generate negative airway pressure;
wherein controlling the phrenic nerve stimulator comprises maintaining the stimulation energy at or above said diaphragmic muscle contraction threshold while the airway is partially or totally occluded.

6. The system of claim 1, wherein the operations further comprise:
receiving or determining, for a given patient, a patient waking-up threshold not sufficient to wake up the patient;
wherein controlling the phrenic nerve stimulator comprises maintaining the stimulation energy below the patient waking-up threshold while the airway is partially or totally occluded.

7. The system of claim 1, wherein the operations further comprise:
establishing whether the patient is resting or sleeping.

8-14. (canceled)

15. The system of claim 7, wherein the operations further comprise: commanding the phrenic nerve stimulator to deliver the stimulation energy or increase delivery of the stimulation energy while the patient is established to be resting or sleeping.

16. The system of claim 1, wherein controlling the phrenic nerve stimulator to deliver the stimulation energy based on the one or more signals is periodically repeated from 6 to 20 times per minute.

17. (canceled)

18. System according to claim 16, wherein controlling the phrenic nerve stimulator to deliver the stimulation energy based on the one or more signals is periodically repeated while the patient is determined to be resting or sleeping.

19. (canceled)

20. The system of claim 1, wherein the stimulation energy is delivered to the phrenic nerve as a train of pulses.

21. The system of claim 20, wherein the operations further comprise controlling one or more pulse train parameters, the one or more pulse train parameters including: 1) a number of individual pulses in the train of pulses, 2) a frequency of individual pulses in the train of pulses, 3) an amplitude of individual pulses in the train of pulses, and/or 4) a duration of individual pulses in the train of pulses.

22-26. (canceled)

27. The system of claim 1, wherein the operations further comprise:
commanding at time intervals, a ramp or a stepwise increase of the stimulation energy.

28. The system of claim 1, wherein the operations further comprise:
receiving or determining a therapeutic range and
controlling the phrenic nerve stimulator to deliver the stimulation energy within said therapeutic range,
wherein the therapeutic range is between a first threshold and a second threshold, higher than the first threshold.

29. (canceled)

30. The system of claim 28, wherein the operations further comprises receiving or determining a plurality of therapeutic ranges for the same patient, the plurality of therapeutic ranges comprising a therapeutic range determined for each respective different posture of the patient during resting or sleeping.

31. The system of claim 27, wherein controlling the phrenic nerve stimulator to deliver the stimulation energy comprises stopping the ramp or stepwise increase of delivered energy based on a determination that the patient is no longer resting or sleeping.

32. (canceled)

33. The system of claim 1, wherein the stimulation energy comprises at least one first stimulation burst applied first at a first energy level sufficient to generate action potentials in a phrenic nerve of the patient and at least one second energy level applied after the first stimulation burst and sufficient to evoke reflex opening of an occluded airway by activation of upper airway muscles.

34. The system of claim 1, wherein the stimulation energy comprises at least one first stimulation burst applied first at a first energy level sufficient to generate action potentials in a phrenic nerve of the patient and further as at least one second stimulation burst at a second energy level sufficient to evoke reflex opening of an occluded airway by potentiation of a mechanoreflex.

35-36. (canceled)

37. The system of claim 1, wherein a first portion of the controller and the nerve stimulator are within an implantable pulse generator and a second portion of the controller is on an external device that is communicatively connected with the first portion of the controller.

38-49. (canceled)

50. A controller for a system configured to treat obstructive sleep apnea (OSA), the system comprising:
a phrenic nerve stimulator configured to deliver stimulation energy to a phrenic nerve in a sleeping patient;
at least one sensor configured to output one or more signals indicative of an occlusion status in an airway; and
wherein the controller is configured to execute a procedure comprising:
receiving the one or more signals, and
controlling the phrenic nerve stimulator to deliver the stimulation energy based on the one or more signals.

51. (canceled)

52. A method for treating obstructive sleep apnea (OSA) by delivering stimulation energy to a phrenic nerve of a sleeping patient:
delivering, via a phrenic nerve stimulator, stimulation energy to a phrenic nerve in a sleeping patient;
receiving, via at least one sensor, one or more signals indicative of an occlusion status in an airway;
controlling the phrenic nerve stimulator to deliver the stimulation energy based on the one or more signals.
